(19) United States
(12) Patent Application Publication
(10) Pub. No.: US 2021/0170102 A1
(43) Pub. Date: Jun. 10, 2021

(54) Utilizing Pressure Measurements to Detect Reuse of Patient Lines

(71) Applicant: BAYER HEALTHCARE LLC, WHIPPANY, NJ (US)

(72) Inventor: SAMANTHA PARKER, PITTSBURGH, PA (US)

(21) Appl. No.: 17/270,102

(22) PCT Filed: Aug. 27, 2019
(86) PCT No.: PCT/US2019/048337

§ 371 Date: Feb. 22, 2021

Related U.S. Application Data
(60) Provisional application No. 62/723,718, filed on Aug. 28, 2018.

(51) Int. Cl. A61M 5/172; A61M 39/24; A61M 5/00; A61M 5/14; A61B 6/00

(52) U.S. Cl. CPC A61M 5/172; A61M 39/24; A61M 5/007; A61M 5/1407; A61M 2205/505; A61M 2005/1402; A61M 2205/52; A61M 2205/18; A61B 6/481

(57) ABSTRACT

A fluid injector system configured to perform an injection protocol in connection with a diagnostic imaging procedure includes a memory for storing a predetermined pressure profile representative of pressure expected to be generated within an exemplary administration line by a priming fluid, and a control device operatively associated with a drive component to pressurize and inject at least one fluid through a subject administration line. The control device includes a processor configured to perform an operation including: actuating the drive component to prime the subject administration line; determining a distinct pressure profile indicative of a measurement of current pressure generated during the priming of the subject administration line; comparing the distinct pressure profile to the predetermined pressure profile; and determining, based on a result of the comparison, whether the subject administration line, prior to priming, contained at least one of a liquid or a gas as the extant fluid.

### CROSS-REFERENCE TO RELATED APPLICATIONS

The present application claims the benefit of U.S. Provisional Patent Application No. 62/723,718 filed Aug. 28, 2018, the disclosure of which is hereby incorporated by reference in its entirety.

### BACKGROUND OF THE DISCLOSURE

### Field of the Disclosure

The present disclosure relates generally to detecting reuse of single-use disposable fluid path set components in fluid injector systems. More specifically, the present disclosure relates to fluid injector systems, computer program products, and methods for utilizing pressure measurements to detect reuse of single-use disposable fluid path set components in such fluid injector systems.

### Description of the Related Art

In many medical diagnostic and therapeutic procedures, a medical practitioner, such as a physician, injects a patient with one or more medical fluids. In recent years, a number of medical fluid delivery systems for pressurized injection of fluids, such as a contrast solution (often referred to simply as “contrast”), a flushing agent or diluent, such as saline, and other medical fluids, have been developed for use in procedures such as angiography, computed tomography (CT), ultrasound, magnetic resonance imaging (MM), positron emission tomography (PET), and other molecular imaging procedures. In general, these medical fluid delivery systems are designed to deliver a preset amount of fluid at a preset flow rate.

In some injection procedures, the medical practitioner places a catheter or needle into a vein or artery of the patient. The catheter or needle is connected to either a manual or an automatic fluid injector system by way of tubing and a connector that interfaces with the fluid injector system. Automatic fluid injector systems typically include at least one syringe connected to at least one fluid injector having, for example, a powered linear piston. The at least one syringe includes, for example, a source of contrast and/or a source of flushing fluid. The medical practitioner enters settings into an electronic control system of the fluid injector for a fixed volume of contrast and/or saline and a fixed rate of injection for each. A single-use disposable set connector and associated tubing are connected to the fluid injector system for delivering one or more fluids to the patient.

To prevent contamination between patients and medical devices, each single-use disposable set connector and associated tubing is ideally replaced between patients. However, users may be inclined to reuse single-use disposable set connectors to save time and cost, an unhygienic and potentially dangerous practice.

While various manual and automatic fluid delivery systems are known in the medical field, improved multi-fluid delivery systems adapted for use in medical diagnostic and therapeutic procedures where one or more fluids are supplied to a patient during such procedures continue to be in demand. In particular, there exists a need for fluid delivery systems and single-use disposable set connectors which encourage and enforce safe and hygienic work practices.

### SUMMARY OF DISCLOSURE

The present disclosure generally relates to a fluid injector system configured to perform an injection protocol in connection with a diagnostic imaging procedure. The fluid injector system may include a memory for storing therein a predetermined pressure profile, wherein the predetermined pressure profile may be representative of pressure expected to be generated within an exemplary administration line by a priming fluid over a course of a priming operation performed on the exemplary administration line during which the priming fluid completely displaces an extant fluid from the exemplary administration line. The fluid injector system further may include a control device operatively associated with at least one drive component configured to pressurize and inject at least one fluid through a subject administration line into a patient. The control device may include at least one processor programmed or configured to perform an operation including: actuating the at least one drive component to prime the subject administration line with the at least one fluid as the priming fluid, determining a distinct pressure profile indicative of a measurement of current pressure generated during the priming of the subject administration line with the at least one fluid over the course of the priming operation performed therewith, comparing the distinct pressure profile to the predetermined pressure profile, and determining, based on a result of the comparison, whether the subject administration line, prior to the priming thereof, contained therein at least one of a liquid as the extant fluid and a gas as the extant fluid.

In accordance with other examples, the exemplary administration line may be one of (i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and (ii) a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation. When the exemplary administration line is the unused administration line, upon the comparison resulting in a correlation within a specified tolerance between the distinct pressure profile and the predetermined pressure profile, the at least one processor is configured to determine that the subject administration line contained the gas as the extant fluid.

In accordance with other examples, the operation may further include: upon the comparison resulting in the correlation within the specified tolerance between the distinct pressure profile and the predetermined pressure profile, generating an alert indicating that the subject administration line had not been used prior to being primed with the at least one fluid during the priming operation. The operation may further include: upon the comparison resulting in the correlation within the specified tolerance between the distinct pressure profile and the predetermined pressure profile, permitting performance of the injection protocol.

In accordance with other examples, the subject administration line, like the exemplary administration line, may include at least one check valve precluding fluid flow in a proximal direction. The comparison resulting in the correlation within the specified tolerance between the distinct pressure profile and the predetermined pressure profile may include at least one of: identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

In accordance with other examples, the exemplary administration line may be the previously used administration line, and upon the comparison resulting in a correlation within a specified tolerance between the distinct pressure profile and the predetermined pressure profile, the at least one processor may be configured to determine that the subject administration line contained the liquid as the extant fluid. The operation may further include: upon the comparison resulting in the correlation within the specified tolerance between the distinct pressure profile and the predetermined pressure profile, generating an alert indicating that the subject administration line had been used prior to being primed with the at least one fluid during the priming operation.

In accordance with other examples, the subject administration line, like the exemplary administration line, may include at least one check valve precluding fluid flow in a proximal direction. The comparison resulting in the correlation within the specified tolerance between the distinct pressure profile and the predetermined pressure profile may include at least one of: identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

In accordance with other examples, the at least one fluid used in the priming of the subject administration line may include at least one of (i) a diluent, (ii) a contrast medium and (iii) a mixture of the contrast medium and the diluent. The determining the distinct pressure profile may include measuring a motor current of the at least one drive component.

In accordance with other examples, the subject administration line, like the exemplary administration line, may include a first check valve precluding fluid flow in a proximal direction and a second check valve precluding flow in the proximal direction. The second check valve may be located distally of the first check valve. During the priming of the subject administration line, the operation further may include: identifying a first pressure inflection point in the distinct pressure profile caused by the at least one fluid having passed through the first check valve in the subject administration line; and identifying at least one of: a second pressure inflection point in the distinct pressure profile caused by the at least one fluid having passed through the second check valve in the subject administration line; and a steady state portion of the distinct pressure profile over which the pressure generated within the subject administration line remains substantially constant. While comparing the distinct pressure profile to the predetermined pressure profile, the operation may further include: determining, based on a result of the comparison, that the administration line is fully primed.

In accordance with other examples, the subject administration line, like the exemplary administration line, may include a single check valve located in a distal end of the subject administration line. The single check valve may preclude fluid flow in a proximal direction. During the priming of the subject administration line, the operation further may include identifying at least one of: a pressure inflection point in the distinct pressure profile caused by the at least one fluid having passed through the single check valve in the subject administration line; and a steady state portion of the distinct pressure profile over which the pressure generated within the subject administration line remains substantially constant. While comparing the distinct pressure profile to the predetermined pressure profile, the operation may further include: determining, based on a result of the comparison, a presence of a fluid path set component connected to a distal end of the administration line.

In accordance with other examples, a computer program product for detecting multiple uses of an administration line using a fluid injector system may be configured to perform an injection protocol in connection with a diagnostic imaging procedure. The computer program product may include non-transitory computer readable media having a memory for storing therein a predetermined pressure profile. The predetermined pressure profile may be representative of pressure expected to be generated within an exemplary administration line by a priming fluid over a course of a priming operation performed on the exemplary administration line during which the priming fluid completely displaces an extant fluid from the exemplary administration line. The non-transitory computer readable media further may include one or more instructions that, when executed by at least one processor, cause the at least one processor to perform an operation including: actuating at least one drive component of the fluid injector system to prime a subject administration line with the priming fluid; determining a distinct pressure profile indicative of a measurement of current pressure generated during the priming of the subject administration line with the at least one fluid over the course of the priming operation performed therewith; comparing the distinct pressure profile to the predetermined pressure profile; and determining, based on a result of the comparison, whether the subject administration line, prior to the priming thereof, contained therein at least one of a liquid as the extant fluid and a gas as the extant fluid.

In accordance with other examples, the exemplary administration line may be one of (i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and (ii) a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation. When the exemplary administration line is the unused administration line, upon the comparison resulting in a correlation within a specified tolerance between the distinct pressure profile and the predetermined pressure profile, the one or more instructions, when executed by the at least one processor, may cause the at least one processor to determine that the subject administration line contained the gas as the extant fluid.

In accordance with other examples, the one or more instructions, when executed by the at least one processor, may cause the at least one processor to perform a further operation including: upon the comparison resulting in the correlation within the specified tolerance between the distinct pressure profile and the predetermined pressure profile, generating an alert indicating that the subject administration line had not been used prior to being primed with the at least one fluid during the priming operation. The one or more instructions, when executed by the at least one processor, may cause the at least one processor to perform a further operation including: upon the comparison resulting in the correlation within the specified tolerance between the distinct pressure profile and the predetermined pressure profile, permitting performance of the injection protocol.

In accordance with other examples, the subject administration line, like the exemplary administration line, may include at least one check valve precluding fluid flow in a proximal direction. The comparison resulting in the correlation within the specified tolerance between the distinct pressure profile and the predetermined pressure profile may include at least one of: identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

In accordance with other examples, the exemplary administration line may be the previously used administration line, and upon the comparison resulting in a correlation within a specified tolerance between the distinct pressure profile and the predetermined pressure profile, the one or more instructions, when executed by the at least one processor, may cause the at least one processor to determine that the subject administration line contained the liquid as the extant fluid. The one or more instructions, when executed by the at least one processor, may cause the at least one processor to perform a further operation including: upon the comparison resulting in the correlation within the specified tolerance between the distinct pressure profile and the predetermined pressure profile, generating an alert indicating that the subject administration line had been used prior to being primed with the at least one fluid during the priming operation.

In accordance with other examples, the subject administration line, like the exemplary administration line, may include at least one check valve precluding fluid flow in a proximal direction. The comparison resulting in the correlation within the specified tolerance between the distinct pressure profile and the predetermined pressure profile may include at least one of: identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and normalizing, with the at least one processor, the distinct pressure profile about a steady state value thereof, normalizing, with the at least one processor, the predetermined pressure profile about a steady state value thereof, and determining, with the at least one processor, that each point along the distinct pressure profile correlates within a specified tolerance to corresponding points on the predetermined pressure profile.

In accordance with other examples, the at least one fluid used in the priming of the subject administration line may include at least one of (i) a diluent, (ii) a contrast medium and (iii) a mixture of the contrast medium and the diluent. The determining the distinct pressure profile may include measuring, with the at least one processor, a motor current of the at least one drive component.

In accordance with other examples, the subject administration line, like the exemplary administration line, may include a first check valve precluding fluid flow in a proximal direction and a second check valve precluding flow in the proximal direction. The second check valve may be located distally of the first check valve. During priming of the subject administration line, the operation further may include: identifying a first pressure inflection point in the distinct pressure profile caused by the at least one fluid having passed through the first check valve in the subject administration line; and identifying at least one of: a second pressure inflection point in the distinct pressure profile caused by the at least one fluid having passed through the second check valve in the subject administration line; and a steady state portion of the distinct pressure profile over which the pressure generated within the subject administration line remains substantially constant. While comparing the distinct pressure profile to the predetermined pressure profile, the operation further may include: determining, based on a result of the comparison, that the administration line is fully primed.

In accordance with other examples, the subject administration line, like the exemplary administration line, may include a single check valve located in a distal end of the subject administration line. The single check valve may preclude fluid flow in a proximal direction. During the priming of the subject administration line, the operation further may include identifying at least one of: a pressure inflection point in the distinct pressure profile caused by the at least one fluid having passed through the single check valve in the subject administration line; and a steady state portion of the distinct pressure profile over which the pressure generated within the subject administration line remains substantially constant. While comparing the distinct pressure profile to the predetermined pressure profile, the operation further may include: determining, based on a result of the comparison, a presence of a fluid path set component connected to a distal end of the administration line.

In accordance with other examples, a method for detecting multiple uses of an administration line using a fluid injector system configured to perform an injection protocol in connection with a diagnostic imaging procedure may include: providing a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line; actuating at least one drive component of the fluid injector system to prime a subject administration line with the priming fluid; determining a distinct pressure profile indicative of a measurement of current pressure generated during the priming of the subject administration line with the at least one fluid over the course of the priming operation performed therewith; comparing the distinct pressure profile to the predetermined pressure profile; and determining, based on a result of the comparison, whether the subject administration line, prior to the priming thereof, contained therein at least one of a liquid as the extant fluid and a gas as the extant fluid.

In accordance with other examples, the disclosure of the present application may be characterized by one or more of the following clauses:

Clause 1: A fluid injector system configured to perform an injection protocol in connection with a diagnostic imaging procedure, the fluid injector system comprising: a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line; a control device operatively associated with at least one drive component configured to pressurize and inject at least one fluid through a subject administration line into a patient, the control device including at least one processor programmed or configured to perform an operation comprising: actuating the at least one drive component to prime the subject administration line with the at least one fluid as the priming fluid; determining a distinct pressure profile indicative of a measurement of current pressure generated during the priming of the subject administration line with the at least one fluid over the course of the priming operation performed therewith; comparing the distinct pressure profile to the predetermined pressure profile; and determining, based on a result of the comparison, whether the subject administration line, prior to the priming thereof, contained therein at least one of a liquid as the extant fluid and a gas as the extant fluid.

Clause 2: The fluid injector system of clause 1, wherein the exemplary administration line is one of: (i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and (ii) a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation.

Clause 3: The fluid injector system of clause 1 or 2, wherein the exemplary administration line is the unused administration line and upon the comparison resulting in a correlation within a specified tolerance between the distinct pressure profile and the predetermined pressure profile, the at least one processor is configured to determine that the subject administration line contained the gas as the extant fluid.

Clause 4: The fluid injector system of any of clauses 1 to 3, wherein the operation further comprises: upon the comparison resulting in the correlation within the specified tolerance between the distinct pressure profile and the predetermined pressure profile, generating an alert indicating that the subject administration line had not been used prior to being primed with the at least one fluid during the priming operation.

Clause 5: The fluid injection system of any of clauses 1 to 4, wherein the operation further comprises: upon the comparison resulting in the correlation within the specified tolerance between the distinct pressure profile and the predetermined pressure profile, permitting performance of the injection protocol.

Clause 6: The fluid injector system of any of clauses 1 to 5, wherein the subject administration line, like the exemplary administration line, comprises at least one check valve precluding fluid flow in a proximal direction; and wherein the comparison resulting in the correlation within the specified tolerance between the distinct pressure profile and the predetermined pressure profile comprises at least one of: identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

Clause 7: The fluid injector system of any of clauses 1 to 6, wherein the exemplary administration line is the previously used administration line and upon the comparison resulting in a correlation within a specified tolerance between the distinct pressure profile and the predetermined pressure profile, the at least one processor is configured to determine that the subject administration line contained the liquid as the extant fluid.

Clause 8: The fluid injector system of any of clauses 1 to 7, wherein the operation further comprises: upon the comparison resulting in the correlation within the specified tolerance between the distinct pressure profile and the predetermined pressure profile, generating an alert indicating that the subject administration line had been used prior to being primed with the at least one fluid during the priming operation.

Clause 9: The fluid injector system of any of clauses 1 to 8, wherein the subject administration line, like the exemplary administration line, comprises at least one check valve precluding fluid flow in a proximal direction; and wherein the comparison resulting in the correlation within the specified tolerance between the distinct pressure profile and the predetermined pressure profile comprises at least one of: identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

Clause 10: The fluid injector system of any of clauses 1 to 9, wherein the at least one fluid used in the priming of the subject administration line comprises at least one of (i) a diluent, (ii) a contrast medium and (iii) a mixture of the contrast medium and the diluent.

Clause 11: The fluid injector system of any of clauses 1 to 10, wherein the determining the distinct pressure profile comprises measuring a motor current of the at least one drive component.

Clause 12: The fluid injector system of any of clauses 1 to 11, wherein the subject administration line, like the exemplary administration line, comprises a first check valve precluding fluid flow in a proximal direction and a second check valve precluding flow in the proximal direction, the second check valve located distally of the first check valve; and wherein, during the priming of the subject administration line, the operation further comprises: identifying a first pressure inflection point in the distinct pressure profile caused by the at least one fluid having passed through the first check valve in the subject administration line; and identifying at least one of: a second pressure inflection point in the distinct pressure profile caused by the at least one fluid having passed through the second check valve in the subject administration line; and a steady state portion of the distinct pressure profile over which the pressure generated within the subject administration line remains substantially constant; whereupon the subject administration line is determined to be fully primed.

Clause 13: The fluid injector system of any of clauses 1 to 12, wherein the subject administration line, like the exemplary administration line, comprises a single check valve located in a distal end of the subject administration line, the single check valve precluding fluid flow in a proximal direction; and wherein, during the priming of the subject administration line, the operation further comprises identifying at least one of: a pressure inflection point in the distinct pressure profile caused by the at least one fluid having passed through the single check valve in the subject administration line; and a steady state portion of the distinct pressure profile over which the pressure generated within the subject administration line remains substantially constant; whereupon the subject administration line at a distal end thereof is determined to have a fluid path set component connected thereto.

Clause 14: A computer program product for detecting multiple uses of an administration line using a fluid injector system configured to perform an injection protocol in connection with a diagnostic imaging procedure, the computer program product comprising: non-transitory computer readable media comprising a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line; the non-transitory computer readable media further comprising one or more instructions that, when executed by at least one processor, cause the at least one processor to perform an operation comprising: actuating at least one drive component of the fluid injector system to prime a subject administration line with the priming fluid; determining a distinct pressure profile indicative of a measurement of current pressure generated during the priming of the subject administration line with the at least one fluid over the course of the priming operation performed therewith; comparing the distinct pressure profile to the predetermined pressure profile; and determining, based on a result of the comparison, whether the subject administration line, prior to the priming thereof, contained therein at least one of a liquid as the extant fluid and a gas as the extant fluid.

Clause 15: The computer program product of clause 14, wherein the exemplary administration line is one of (i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation.

Clause 16: The computer program product of clause 14 or 15, wherein the exemplary administration line is the unused administration line and upon the comparison resulting in a correlation within a specified tolerance between the distinct pressure profile and the predetermined pressure profile, the one or more instructions, when executed by the at least one processor, cause the at least one processor to determine that the subject administration line contained the gas as the extant fluid.

Clause 17: The computer program product of any of clauses 14 to 16, wherein the one or more instructions, when executed by the at least one processor, cause the at least one processor to perform a further operation comprising: upon the comparison resulting in the correlation within the specified tolerance between the distinct pressure profile and the predetermined pressure profile, generating an alert indicating that the subject administration line had not been used prior to being primed with the at least one fluid during the priming operation.

Clause 18: The computer program product of any of clauses 14 to 17, wherein the one or more instructions, when executed by the at least one processor, cause the at least one processor to perform a further operation comprising: upon the comparison resulting in the correlation within the specified tolerance between the distinct pressure profile and the predetermined pressure profile, permitting performance of the injection protocol.

Clause 19: The computer program product of any of clauses 14 to 18, wherein the subject administration line, like the exemplary administration line, comprises at least one check valve precluding fluid flow in a proximal direction; and wherein the comparison resulting in the correlation within the specified tolerance between the distinct pressure profile and the predetermined pressure profile comprises at least one of: identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

Clause 20: The computer program product of any of clauses 14 to 19, wherein upon the comparison resulting in a correlation within a specified tolerance between the distinct pressure profile and the predetermined pressure profile, the one or more instructions, when executed by the at least one processor, cause the at least one processor to determine that the subject administration line contained the liquid as the extant fluid.

Clause 21: The computer program product of any of clauses 14 to 20, wherein the one or more instructions, when executed by the at least one processor, cause the at least one processor to perform a further operation comprising: upon the comparison resulting in the correlation within the specified tolerance between the distinct pressure profile and the predetermined pressure profile, generating an alert indicating that the subject administration line had been used prior to being primed with the at least one fluid during the priming operation.

Clause 22: The computer program product of any of clauses 14 to 21, wherein the subject administration line, like the exemplary administration line, comprises at least one check valve precluding fluid flow in a proximal direction; and wherein the comparison resulting in the correlation within the specified tolerance between the distinct pressure profile and the predetermined pressure profile comprises at least one of: identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line; normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and normalizing, with the at least one processor, the distinct pressure profile about a steady state value thereof, normalizing, with the at least one processor, the predetermined pressure profile about a steady state value thereof, and determining, with the at least one processor, that each point along the distinct pressure profile correlates within a specified tolerance to corresponding points on the predetermined pressure profile.

Clause 23: The computer program product of any of clauses 14 to 22, wherein the at least one fluid used in the priming of the subject administration line comprises at least one of (i) a diluent, (ii) a contrast medium and (iii) a mixture of the contrast medium and the diluent.

Clause 24: The computer program product of any of clauses 14 to 23, wherein the determining the distinct pressure profile comprises measuring, with the at least one processor, a motor current of the at least one drive component.

Clause 25: The computer program product of any of clauses 14 to 24, wherein the subject administration line, like the exemplary administration line, comprises a first check valve precluding fluid flow in a proximal direction and a second check valve precluding flow in the proximal direction, the second check valve located distally of the first check valve; and wherein, during priming of the subject administration line, the operation further comprises: identifying a first pressure inflection point in the distinct pressure profile caused by the at least one fluid having passed through the first check valve in the subject administration line; and identifying at least one of: a second pressure inflection point in the distinct pressure profile caused by the at least one fluid having passed through the second check valve in the subject administration line; and a steady state portion of the distinct pressure profile over which the pressure generated within the subject administration line remains substantially constant; whereupon the subject administration line is determined to be fully primed.

Clause 26: The computer program product of any of clauses 14 to 25, wherein the subject administration line, like the exemplary administration line, comprises a single check valve located in a distal end of the subject administration line, the single check valve precluding fluid flow in a proximal direction; and wherein, during the priming of the subject administration line, the operation further comprises identifying at least one of: a pressure inflection point in the distinct pressure profile caused by the at least one fluid having passed through the single check valve in the subject administration line; and a steady state portion of the distinct pressure profile over which the pressure generated within the subject administration line remains substantially constant; whereupon the subject administration line at a distal end thereof is determined to have a fluid path set component connected thereto.

Clause 27: A method for detecting multiple uses of an administration line using a fluid injector system configured to perform an injection protocol in connection with a diagnostic imaging procedure, the method comprising: providing a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line; actuating at least one drive component of the fluid injector system to prime a subject administration line with the priming fluid; determining a distinct pressure profile indicative of a measurement of current pressure generated during the priming of the subject administration line with the at least one fluid over the course of the priming operation performed therewith; comparing the distinct pressure profile to the predetermined pressure profile; and determining, based on a result of the comparison, whether the subject administration line, prior to the priming thereof, contained therein at least one of a liquid as the extant fluid and a gas as the extant fluid.

These and other features and characteristics of fluid injector systems, computer program products, and methods of operation and functions of the related elements of structures and the combination of parts and economies of manufacture, will become more apparent upon consideration of the following description and the appended claims with reference to the accompanying drawings, all of which form a part of this specification. It is to be expressly understood, however, that the drawings are for the purpose of illustration and description only.

### BRIEF DESCRIPTION OF THE DRAWINGS

FIG. 1 is a perspective view of a multi-fluid delivery system, according to one example;

FIG. 2 is a schematic view of various fluid paths within the multi-fluid delivery system of FIG. 1;

FIG. 3A is a perspective view of a connection interface prior to connecting a single-use disposable set connector with a multi-fluid delivery system;

FIG. 3B is a perspective view of the connection interface of FIG. 3A showing the single-use disposable set connector connected with the multi-fluid delivery system;

FIG. 4A is a perspective view of a single-use disposable set connector in accordance with one example;

FIG. 4B is a cross-sectional view of the single-use disposable set connector shown in FIG. 4A;

FIG. 4C is a cross-sectional view of the single-use disposable set connector shown in FIG. 4A connected to a port of a multi-fluid delivery system;

FIG. 5 is a perspective view of the single-use disposable set connector shown in FIG. 4C with a portion of the multi-fluid delivery system and the multi-patient disposable set cutaway;

FIG. 6 is a schematic view of an electronic control system of a multi-fluid injection system in accordance with another example;

FIG. 7 is a graphical representation of a predetermined pressure profile and a distinct pressure profile representative of the single-use disposable set connector of FIGS. 4A-5;

FIG. 8 is a step sequence diagram of a method of determining the nature of an extant fluid in the single-use disposable set connector of FIGS. 4A-5;

FIG. 9 is a graphical representation of the predetermined pressure profile and the distinct pressure profile of FIG. 7 normalized about steady state portions thereof;

FIG. 10 is a step sequence diagram of a method of determining that the single-use disposable set connector of FIGS. 4A-5 is fully primed;

FIG. 11 is a graphical representation of the predetermined pressure profile of FIG. 7 and a modified distinct pressure profile representative of the single-use disposable set connector of FIGS. 4A-5 having an additional fluid path set component attached thereto;

FIG. 12 is a graphical representation of the predetermined pressure profile of FIG. 7 and various predetermined pressure profiles representative of the single-use disposable set connector of FIGS. 4A-5 at various ages and use states; and

FIG. 13 is a graphical representation of the predetermined pressure profile of FIG. 7 and various predetermined pressure profiles representative of the single-use disposable set connector of FIGS. 4A-5 coupled with varying extant fluids in a multi-patient disposable set of the multi-fluid delivery system of FIG. 1.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe, a single-use disposable set connector, and/or a fluid path set component, the term “proximal” refers to a portion of the syringe, the single-use disposable set connector, and/or the fluid path set component nearest to an injector when the syringe, the single-use disposable set connector, and/or the fluid path set component is oriented for connecting to the injector. The term “distal” refers to a portion of the syringe, the single-use disposable set connector, and/or the fluid path set component farthest away from the injector when the syringe, the single-use disposable set connector, and/or the fluid path set component is oriented for connecting to the injector.

As used herein, the term “correlation” and derivatives thereof refers to an observed and/or calculated association(s) between data. Correlation may include, for example, a relative difference between two or more data points, a statistical relationship between two or more data sets, and combinations thereof. As used herein, the term “specified tolerance” refers to a predetermined percentage difference, a predetermined standard deviation, a predetermined statistical correlation coefficient, and/or the like. For example, a first value may exhibit correlation within a specified tolerance of a second value if the first value is within a predetermined percentage difference (e.g., within 10%) of the second value. Similarly, a data point may exhibit correlation within a specified tolerance of a data set if the data point falls within a predetermined standard deviation (e.g., within one standard deviation) of the data set. Similarly, a first curve may exhibit correlation within a specified tolerance of a second curve if the first curve includes specific features (e.g. inflection points) within a predetermined range (e.g., within 10 sampling time intervals) of like features of the second curve. Similarly, a first curve may exhibit correlation within a specified tolerance of a second curve if an area under the first curve is within a predetermined percentage difference (e.g., within 10%) of an area under the second curve.

As used herein, the term “normalize” and derivatives thereof refers to adjusting individual values of a data set to a common scale. For example, normalizing may refer to dividing all values of a data set by a value corresponding to a steady state condition, such that each value of the normalized data set is referenced to the steady state condition.

As used herein, the term “and/or” refers to both or either of two stated possibilities. For example, when used with reference to “first and/or second predetermined pressure profile”, this phrase refers to a combination of both of the first and the second predetermined pressure profile, or one of the first predetermined pressure profile and the second predetermined pressure profile.

All numbers used in the specification and claims are to be understood as being modified in all instances by the term “about”. The terms “about”, “approximately”, and “substantially” means a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

Although the present disclosure is described primarily in the context of the MEDRAD® Centargo CT Injection System, it will be apparent to persons of ordinary skill in the art that the present disclosure can be applied to a variety of injection systems inclusive of their associated disposables (e.g., syringes, tubing, etc.). Examples of such injection systems include the MEDRAD® Stellant CT Injection System, the MEDRAD® Stellant FLEX CT Injection System, the MEDRAD® MRXperion MR Injection System and the MEDRAD® Mark 7 Arterion Injection System offered by Bayer HealthCare LLC.

Referring now to the drawings in which like reference characters refer to like parts throughout the several views thereof, the present disclosure in some aspects and examples thereof is generally directed to a multi-fluid medical injector/injection system 100 (hereinafter “fluid injector system 100”) having a multi-patient disposable set (MUDS) 130 configured for delivering fluid to a patient using a single-use disposable set (SUDS) 190 connector. The fluid injector system 100 includes multiple components as individually described herein. Generally, the fluid injector system 100 has a powered injector or other administration device and a fluid delivery set intended to be associated with the injector to deliver one or more fluids from one or more multi-dose containers under pressure into a patient, as described herein. The various devices, components, and features of the fluid injector system 100 and the fluid delivery set associated therewith are likewise described in detail herein.

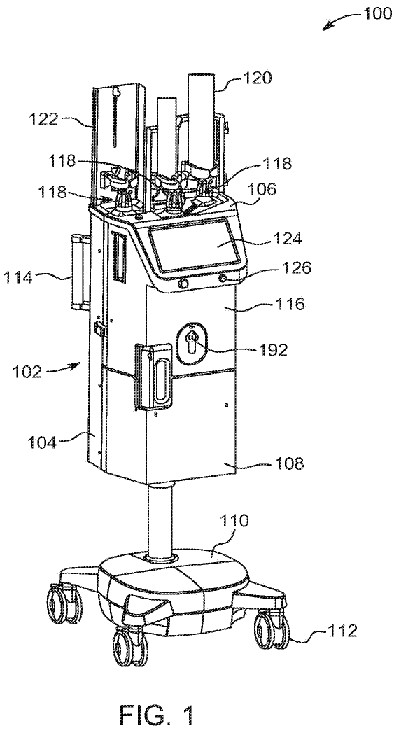

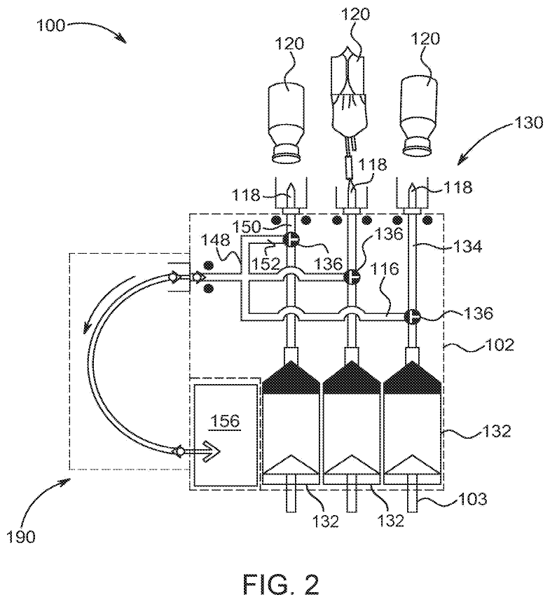

With reference to FIG. 1, the fluid injector system 100 includes an injector housing 102 having opposed lateral sides 104, a distal or upper end 106, and a proximal or lower end 108. In some examples, the housing 102 may be supported on a base 110 having one or more wheels 112 for rotatable and movable support of the housing 102 on a floor surface. The one or more wheels 112 may be lockable to prevent the housing 102 from inadvertently moving once positioned at a desired location. At least one handle 114 may be provided to facilitate moving and positioning the fluid injector system 100. In other examples, the housing 102 may be removably or non-removably secured to a fixed surface, such as a floor, ceiling, wall, or other structure. The housing 102 encloses the various mechanical drive components, electrical and power components necessary to drive the mechanical drive components, and control components, such as electronic memory and electronic control devices (hereinafter electronic control device(s)), used to control operation of reciprocally movable piston elements 103 (shown in FIG. 2) associated with the fluid injector system 100 described herein. Such piston elements 103 may be reciprocally operable via electro-mechanical drive components such as a ball screw shaft driven by a motor, a voice coil actuator, a rack-and-pinion gear drive, a linear motor, and the like. In some examples, at least some of the mechanical drive components, electrical and power components, and control components may be provided on the base 110.

With continued reference to FIG. 1, the fluid injector system 100 has at least one door 116 that encloses at least some of the MUDS, the mechanical drive components, electrical and power components, and control components. The door 116 is desirably movable between an open position and a closed position (shown in FIG. 1). In some examples, the door 116 may be lockable.

The fluid injector system 100 further includes at least one bulk fluid connector 118 for connection with at least one bulk fluid source 120. In some examples, a plurality of bulk fluid connectors 118 may be provided. For example, as shown in FIG. 1, three bulk fluid connectors 118 may be provided in a side-by-side or other arrangement. In some examples, the at least one bulk fluid connector 118 may be a spike configured for removably connecting to the at least one bulk fluid source 120, such as a vial, a bottle, or a bag. The at least one bulk fluid connector 118 may have a reusable or non-reusable interface with each new bulk fluid source 120. The at least one bulk fluid connector 118 may be formed on the multi-patient disposable set, as described herein. The at least one bulk fluid source 120 may be configured for receiving a medical fluid, such as saline, contrast solution, or other medical fluid, for delivery to the fluid injector system 100. The housing 102 may have at least one support member 122 for supporting the at least one bulk fluid source 120 once it is connected to the fluid injector system 100.

With continued reference to FIG. 1, the fluid injector system 100 includes one or more user interfaces 124, such as a graphical user interface (GUI) display window. The user interface 124 may display information pertinent to a fluid injection procedure involving the fluid injector system 100, such as current flow rate, fluid pressure, and volume remaining in the at least one bulk fluid source 120 connected to the fluid injector system 100 and may be a touch screen GUI that allows an operator to input commands and/or data for operation of the fluid injector system 100. While the user interface 124 is shown on the injector housing 102, such user interface 124 may also be in the form of a remote display that is wired or wirelessly linked to the housing 102 and control and mechanical elements of fluid injector system 100. In some examples, the user interface 124 may be a tablet computer that is detachably connected to the housing 102 and is in wired or wirelessly linked communication with the housing 102 and control and mechanical elements of the fluid injector system 100. Additionally, the fluid injector system 100 and/or user interface 124 may include at least one control button 126 for tactile operation by an attendant operator of the fluid injector system 100. In certain examples, the at least one control button 126 may be part of a keyboard for inputting commands and/or data by the operator. The at least one control button 126 may be hard-wired to the electronic control device(s) associated with the fluid injector system 100 to provide direct input to the electronic control device(s). The at least one control button 126 may also be a graphical part of the user interface 124, such as a touch screen. In either arrangement, the at least one control button 126 desirably provides certain individual control features to the attendant operator of the fluid injector system 100, such as, but not limited to: (1) acknowledging that a multi-patient disposable set has been loaded or unloaded; (2) locking/unlocking the multi-patient disposable set; (3) filling/purging the fluid injector system 100; (4) inputting information and/or data related to the patient and/or injection procedure; (5) preloading the fluid injector system 100; and (6) initiating/stopping an injection procedure. The user interface 124 and/or any electronic processing units associated with the fluid injector system 100 may be wired or wirelessly connected to an operation and/or data storage system such as a hospital network system.

With reference to FIG. 2, the fluid injector system 100 includes a MUDS 130 that is removably connected to the fluid injector system 100 for delivering one or more fluids from the one or more bulk fluid sources 120 to the patient. Examples and features of the MUDS are further described in International Patent Application Publication No. WO 2016/112163, filed on Jan. 7, 2016 and entitled “Multiple Fluid Delivery System with Multi-Use Disposable Set and Features Thereof”, the disclosure of which is incorporated herein by reference in its entirety. The MUDS 130 may include one or more syringes or pumps 132. In some examples, the number of syringes 132 may correspond to the number of bulk fluid sources 120. For example, with reference to FIG. 2, the MUDS 130 has three syringes 132 in a side-by-side arrangement such that each syringe 132 is fluidly connectable to one or more of the bulk fluid sources 120. In some examples, one or two bulk fluid sources 120 may be connected to one or more syringes 132 of the MUDS 130. Each syringe 132 may be fluidly connectable to one of the bulk fluid sources 120 by a corresponding bulk fluid connector 118 and an associated MUDS fluid path 134. The MUDS fluid path 134 may have a spike element that connects to the bulk fluid connector 118. In some examples, the bulk fluid connector 118 may be provided directly on the MUDS 130.

With further reference to FIG. 2, the MUDS 130 is removably connectable to the housing 102 of the fluid injector system 100. As will be appreciated by one having ordinary skill in the art, it may be desirable to construct at least a portion of the MUDS 130 from a clear medical grade plastic in order to facilitate visual verification that a fluid connection has been established with the fluid injector system 100. Visual verification is also desirable for confirming that no air bubbles are present within various fluid connections. Alternatively, at least a portion of the MUDS 130 and/or door 116 may include windows (not shown) for visualization of the connection between various components. Various optical sensors (not shown) may also be provided to detect and verify the connections. Additionally, various lighting elements (not shown), such as light emitting diodes (LEDs), may be provided to actuate one or more optical sensors and indicate that a suitable connection has been established between the various components.

With specific reference to FIG. 2, a schematic view of various fluid paths of the fluid injector system 100 is provided. The MUDS 130 may include one or more valves 136, such as stopcock valves, for controlling which medical fluid or combinations of medical fluids are withdrawn from the multi-dose bulk fluid source 120 and/or are delivered to a patient through each syringe 132. In some examples, the one or more valves 136 may be provided on a distal end of the plurality of syringes 132 or on a manifold 148. The manifold 148 may be in fluid communication via valves 136 and/or syringes 132 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the one or more syringes 132 or it may be delivered from the one or more syringes 132. In a first position, such as during the filling of the syringes 132, the one or more valves 136 are oriented such that fluid flows from the bulk fluid source 120 into the desired syringe 132 through a fluid inlet line 150, such as a MUDS fluid path. During the filling procedure, the one or more valves 136 are positioned such that fluid flow through one or more fluid outlet lines 152 or the manifold 148 is blocked. In a second position, such as during a fluid delivery procedure, fluid from one or more syringes 132 is delivered to the manifold 148 through the one or more fluid outlet lines 152 or syringe valve outlet ports. During the delivery procedure, the one or more valves 136 are positioned such that fluid flow through one or more fluid inlet lines 150 is blocked. The one or more valves 136, fluid inlet lines 150, and/or fluid outlet lines 152 may be integrated into the manifold 148. The one or more valves 136 may be selectively positioned to the first or second position by manual or automatic handling. For example, the operator may position the one or more valves 136 into the desired position for filling or fluid delivery. In other examples, at least a portion of the fluid injector system 100 is operable for automatically positioning the one or more valves 136 into a desired position for filling or fluid delivery based on input by the operator, as described herein.

With continued reference to FIG. 2, in some examples, the fluid outlet line 152 may also be connected to a waste reservoir 156 of the fluid injector system 100. The waste reservoir 156 is desirably separate from the syringes 132 to prevent contamination. In some examples, the waste reservoir 156 is configured to receive waste fluid expelled from the syringes 132 during, for example, a flushing, priming, or preloading operation. The waste reservoir 156 may be removable from the housing 102 in order to dispose of the contents of the waste reservoir 156. In other examples, the waste reservoir 156 may have a draining port (not shown) for emptying the contents of the waste reservoir 156 without removing the waste reservoir 156 from the housing 102. In some examples, the waste reservoir 156 is provided as a separate component from the MUDS 130.

Having generally described the components of the fluid injector system 100 and the MUDS 130, the structure and method of use of a single-use disposable set (SUDS) 190 and its interaction with MUDS 130 will now be described. Hereinafter, the SUDS 190 may be referred to as the administration line.

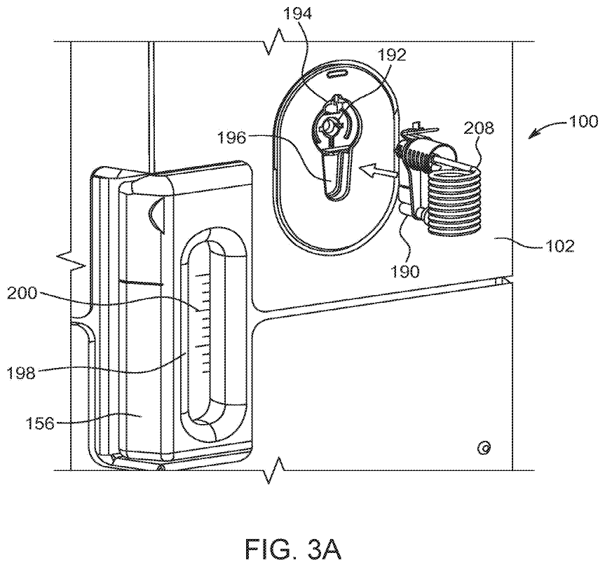

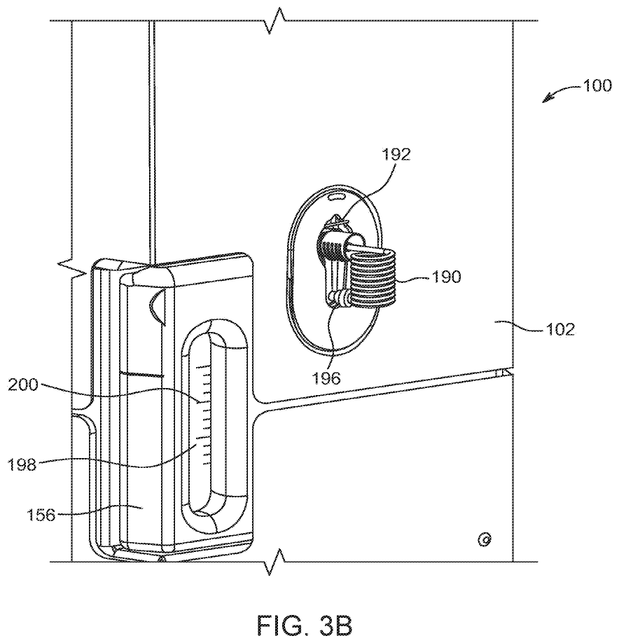

With reference to FIGS. 3A and 3B, the fluid injector system 100 has a connection port 192 that is configured to form a releasable fluid connection with at least a portion of the SUDS 190. In some examples, the connection port 192 may be formed on the MUDS 130. The connection port 192 may be shielded by at least a portion of the housing 102 of the fluid injector system 100. For example, recessing the connection port 192 within the interior of the housing 102 may preserve the sterility of the connection port 192 by preventing or limiting a user or patient from touching and contaminating the portions of the connection port 192 that contact the fluid to be injected into the patient. In some examples, the connection port 192 is recessed within an opening 194 formed on the housing 102 of the fluid injector system 100, or the connection port 192 may have a shielding structure (not shown) that surrounds at least a portion of the connection port 192. In other examples, the connection port 192 may be formed directly on the housing 102 and connected to the MUDS 130 by a fluid path (not shown). As described herein, the SUDS 190 may be connected to the connection port 192, formed on at least a portion of the MUDS 130 and/or the housing 102. Desirably, the connection between the SUDS 190 and the connection port 192 is a releasable connection to allow the SUDS 190 to be selectively disconnected from the connection port 192 (FIG. 3A) and connected to the connection port 192 (FIG. 3B). In some examples, the SUDS 190 may be disconnected from the connection port 192 and disposed after each fluid delivery procedure, and a new SUDS 190 may be connected to the connection port 192 for a subsequent fluid delivery procedure.

With continued reference to FIGS. 3A and 3B, a waste inlet port 196 may be provided separately from the connection port 192. The waste inlet port 196 is in fluid communication with the waste reservoir 156. In some examples, the waste reservoir 156 is provided separately from the SUDS 190 such that the fluid from the waste inlet port 196 can be delivered to the waste reservoir 156. At least a portion of the SUDS 190 may be releasably connected to or associated with the waste inlet port 196 for introducing waste fluid into the waste reservoir 156 during, for example, a priming operation that expels air from the SUDS 190. The waste reservoir 156 may have a viewing window 198 with indicia 200, such as graduated markings, that indicate the fill level of the waste reservoir 156.

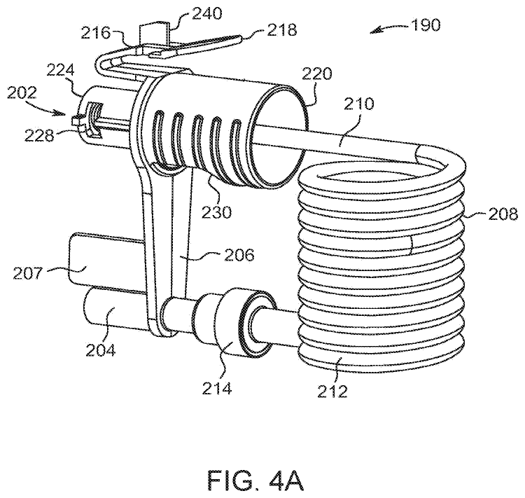

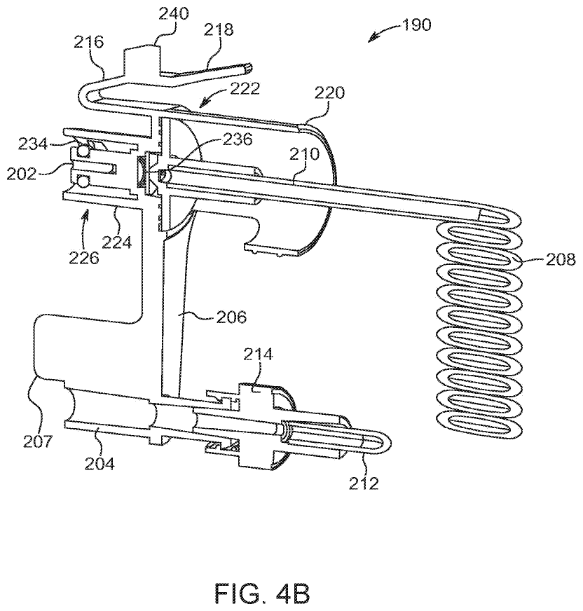

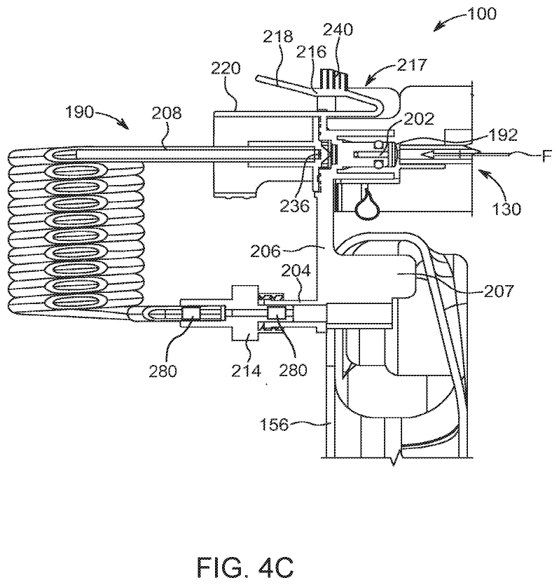

With reference to FIG. 4A, the SUDS 190 has a fluid inlet port 202 that is configured for releasable connection with the connection port 192 (shown in FIG. 3A). The fluid inlet port 202 receives fluid delivered from the fluid injector system 100. The fluid inlet port 202 is desirably a hollow, tubular structure, as shown in FIG. 4B. The SUDS 190 further has a waste outlet port 204 that is configured for releasable connection or association with the waste inlet port 196 (shown in FIG. 3A). The waste outlet port 204 receives waste fluid and delivers such waste fluid to the waste reservoir 156 during, for example, a priming or flushing operation of the SUDS 190. The waste outlet port 204 is desirably a hollow, tubular structure, as shown in FIG. 4B. The waste outlet port 204 may be connected to, inserted into, or located in the waste inlet port 196 so that the waste fluid may flow through the waste inlet port 202 and continue into the waste reservoir 156. The fluid inlet port 202 and the waste outlet port 204 may be spaced apart from each other by a spacer 206. In some examples, the spacer 206 is dimensioned to position the fluid inlet port 202 and the waste outlet port 204 for alignment with the connection port 192 and the waste inlet port 196, respectively. It is noted that the SUDS 190 is shown in FIG. 4A in a state after removal from packaging (not shown). Prior to use, the SUDS 190 is desirably packaged in a pre-sterilized, sealed package that protects the SUDS 190 from contamination with airborne or surface-borne contaminants. Alternatively, the sealed package and the SUDS 190 may be sterilized after packaging.

The SUDS 190 desirably has an asymmetrical structure, so that the user can only attach the SUDS 190 to the MUDS 130 in one orientation. In this manner, the user is prevented from attaching the fluid inlet port 202 to the waste inlet port 196. In some examples, a fin 207 may be provided on at least a portion of the SUDS 190 to prevent erroneous insertion of the SUDS 190 in the connection port 192. In certain examples, the fin 207 may be formed on the spacer 206 proximate to the waste outlet port 204. In this manner, the fin 207 may interfere with the incorrect insertion of the SUDS 190 into the connection port 192. Structures and shapes other than the fin 207 may be used to prevent erroneous insertion of the SUDS 190 into the connection port 192.

In some examples, tubing 208, may be connected at its proximal end 210 to the fluid inlet port 202. The tubing 208 is configured to deliver fluid received from the fluid inlet port 202. The distal end 212 of the tubing 208 may have a connector 214, which may include a one-way check valve, that is configured for connection with the waste outlet port 204 or a fluid path connected to the patient (not shown). The tubing 208 may be made from a flexible material, such as a medical grade plastic material, that allows the tubing 208 to be coiled. The connector 214 may be a luer-lock connector (either a male luer-lock connector or a female luer-lock connector depending on the desired application) or other medical connector configuration. In some examples, the connector 214 may include a one-way check valve 280 therein, as shown in FIGS. 4B and 4C, to prevent backflow of fluid into the tubing 208 from a catheter or other component attached to the connector 214.

With continued reference to FIG. 4A, the SUDS 190 may have a locking tab 216 that is configured for selectively locking the SUDS 190 with the fluid injector system 100 depending on the engagement of the locking tab 216 with at least a portion of the fluid injector system 100. In some examples, the locking tab 216 may be a flexible tab that is deflectable between an engaged position and a disengaged position by deflecting at least a portion of the locking tab 216. The locking tab 216 may have a pressing surface 218 that, when pressed, causes the locking tab 216 to be deflected from the engaged position to the disengaged position for insertion and removal of the SUDS 190 from the fluid injector system 100. In some examples, the locking tab 216 may be configured for releasable locking engagement with a receiving slot 217 on the MUDS 130 (shown in FIG. 4C).

With reference to FIG. 4B, the SUDS 190 may have a first annular skirt 224 extending circumferentially around a proximal end 226 of the fluid inlet port 202 and a second annular skirt 220 extending circumferentially around a distal end 222 of the fluid inlet port 202. The first and second annular skirts 224, 220 surround the fluid inlet port 202 to prevent inadvertent contact and contamination. The first annular skirt 224 may have one or more recesses 228 (shown in FIG. 4A) extending through a sidewall thereof. The one or more recesses 228 may provide a locking interface with a corresponding locking element (not shown) on the fluid injector system 100. The second annular skirt 220 may have at least one indentation 230 (shown in FIG. 4A) to facilitate grasping and handling of the SUDS 190. In some examples, the second annular skirt 220 may have a textured surface having one or more ribs to facilitate gripping and handling of the SUDS 190.

With continued reference to FIG. 4B, at least one annular seal 234 may be provided around the proximal end 226 of the fluid inlet port 202. The at least one annular seal 234 may seal the fluid inlet port 202 to prevent fluid from leaking through the SUDS 190. The at least one annular seal 234 may provide a fluid seal between the SUDS 190 and the MUDS 130 when they are fluidly connected with one another to allow fluid to flow from the MUDS 130 to the SUDS 190 without leaking. A one-way check valve 236 may be provided within a lumen of the fluid inlet port 202 to prevent fluid from flowing in a reverse direction from the SUDS 190 into the MUDS 130.

With reference to FIG. 4C, the SUDS 190 shown in FIG. 4A is shown connected to the fluid injector system 100. While FIG. 4C illustrates the connection port 192 formed on the MUDS 130, in other examples, the connection port 192 may be formed on a portion of the housing 102 (shown in FIG. 1). The fluid inlet port 202 of the SUDS 190 is connected to the connection port 192 to establish a fluid path in a direction of arrow F shown in FIG. 4C. Fluid passing through the fluid inlet port 202 flows through the one-way valve 236 and into the tubing 208. Any fluid that may drip from the interface between the fluid inlet port 202 and the connection port 192 is collected in the waste reservoir 156. The waste reservoir 156 may be shaped to collect any fluid that may drip from the SUDS 190 when it is removed from the MUDS 130. Additionally, when the SUDS 190 is connected to the connection port 192, the outlet of the waste outlet port 204 is positioned within the waste inlet port 196 such that waste fluid from the tubing 208 may be discharged into the waste reservoir 156. The spacer 206 may define an insertion stop surface to define the depth of insertion of the SUDS 190 into the connection port 192.

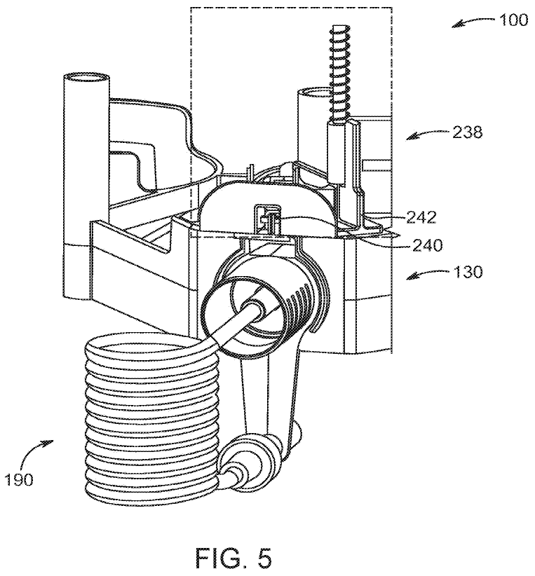

With reference to FIG. 5, the fluid injector system 100 may have a sensor system 238 adapted to identify when the SUDS 190 is in fluid communication with the MUDS 130. The sensor system 238 may include at least one sensing element, such as a sensor fin 240, on the SUDS 190 and a corresponding sensor 242 on the fluid injector system 100 or MUDS 130. The sensor 242 may be configured to detect the presence and absence of the at least one sensor fin 240 or other sensing element. In some examples, the sensing element, such as the at least one sensor fin 240, is formed on the locking tab 216 of the SUDS 190, such as shown in FIG. 4A. In other examples, the sensing element, such as the at least one sensor fin 240, may be formed on any portion of the SUDS 190. The sensor 242 may be an optical sensor that is seated and secured within a respective mount formed on the housing 102 of the fluid injector system 100. As will be appreciated by those versed in the field of powered medical fluid injectors, the sensor 242 may be electronically coupled to an electronic control device used to discretely control operation of the fluid injector system, such as the operation of the one or more piston elements, based, at least in part, on input from the sensor 242. The sensing element, such as the sensor fin 240, may have one or more reflective surfaces that reflect visible or infrared light to be detected by the sensor 242. In other examples, mechanical interaction between the sensing element and the sensor 242 may be used.

In some examples, the SUDS 190 may further include reuse prevention features. For example, the SUDS 190 may include one or more breakable sensor elements, tabs, or structures that fold or break when the SUDS 190 is removed from the MUDS 130. Absence of these features may prevent reinsertion and reuse of the SUDS 190 after removal. In this manner, it can be assured that the SUDS 190 is only used for one fluid delivery procedure.

Other examples and features of the SUDS 190 are described in U.S. Patent Application Publication No. 2016/0331951, filed Jul. 7, 2016 and entitled “Single-Use Disposable Set Connector”, the disclosure of which is incorporated herein by reference in its entirety.

Having generally described the components of the fluid injector system 100, the MUDS 130, and the SUDS 190, a method of operation of using the SUDS 190 will now be described in detail. In use, a medical technician or user removes the disposable SUDS 190 from its packaging (not shown) and inserts the fluid inlet port 202 into the connection port 192 on the MUDS 130. As described above, the SUDS 190 must be inserted in the correct orientation such that the fluid inlet port 202 is aligned for connection with the connection port 192 and the waste outlet port 204 is aligned for connection with the waste inlet port 196. The SUDS 190 may be secured to the MUDS 130 by inserting the locking tab 216 into the receiving slot 217 on the MUDS 130. Once the SUDS 190 is securely connected to the MUDS 130, for example as sensed by the sensor 242, the fluid injector system 100 (shown in FIG. 1) draws fluid into one or more of the plurality of syringes 132 of the MUDS 130 and performs an automatic priming or flushing operation for removing air from the MUDS 130 and the SUDS 190. During such priming or flushing operation, fluid from the MUDS 130 is injected through the connection port 192 and into the tubing 208 of the SUDS 190. The fluid flows through the tubing 208, the connector 214 and through the waste outlet port 204 and into the waste reservoir 156. Once the automatic priming or flushing operation is completed, the tubing 208 may optionally be preloaded with an injection protocol by injecting fluid(s) from the MUDS 130 through the connection port 192. Additional details of the preloading operation will be described later in greater detail. After the automatic priming or flushing operation and, optionally, the preloading operation are completed, the medical technician disconnects the connector 214 from the waste outlet port 204. The connector 214 may then be connected to the patient via a catheter, vascular access device, needle, or additional fluid path set to facilitate fluid delivery to the patient. Once the fluid delivery is completed, the SUDS 190 is disconnected from the patient and the MUDS 130 by disengaging the locking tab 216 of the SUDS 190 from the receiving slot 217 on the MUDS 130. The medical technician may then dispose of the SUDS 190. In certain examples, removing the SUDS 190 from the MUDS 130 causes reuse prevention features (not shown) to activate, thereby preventing reinsertion and reuse of the SUDS 190.

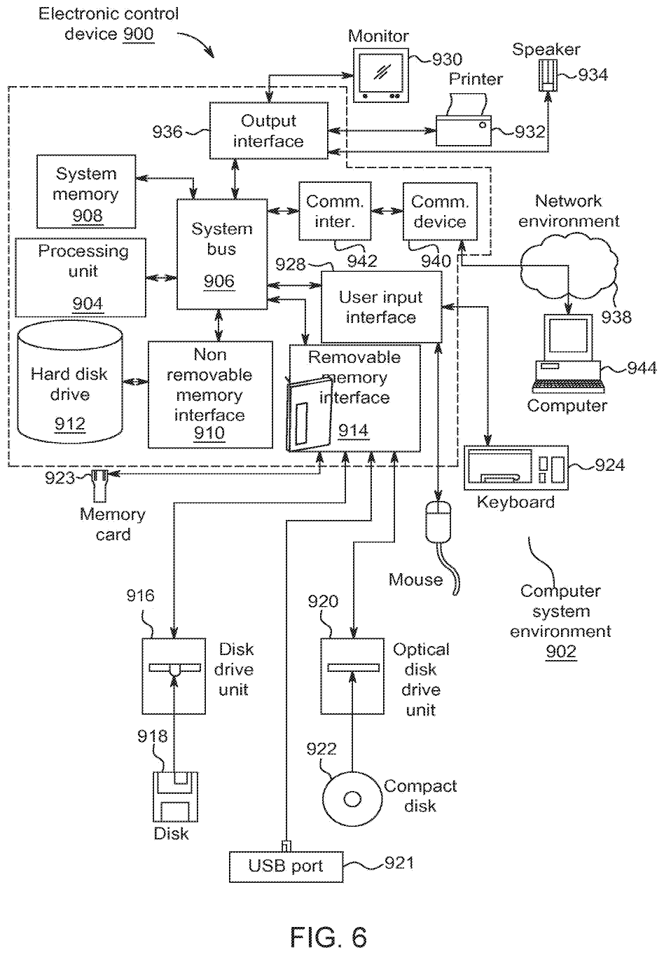

With reference to FIG. 6, an electronic control device 900 may be associated with the fluid injector system 100 to control the filling and delivery operations. In some examples, the electronic control device 900 may control the operation of various valves, piston members, and other elements to effect a desired filling or delivery procedure. For example, the electronic control device 900 may include a variety of discrete computer-readable media components. For example, this computer-readable media may include any media that can be accessed by the electronic control device 900, such as volatile media, non-volatile media, removable media, non-removable media, transitory media, non-transitory media, etc. As a further example, this computer-readable media may include computer storage media, such as media implemented in any method or technology for storage of information, such as computer-readable instructions, data structures, program modules, or other data; random access memory (RAM), read-only memory (ROM), electrically erasable programmable read-only memory (EEPROM), flash memory, or other memory technology; CD-ROM, digital versatile disks (DVDs), or other optical disk storage; magnetic cassettes, magnetic tape, magnetic disk storage, or other magnetic storage devices; or any other medium which can be used to store the desired information and which can be accessed by the electronic control device 900. Further, this computer-readable media may include communications media, such as computer-readable instructions, data structures, program modules, or other data in a modulated data signal, such as a carrier wave or other transport mechanism and include any information delivery media, wired media (such as a wired network and a direct-wired connection), and wireless media (such as acoustic signals, radio frequency signals, optical signals, infrared signals, biometric signals, bar code signals, etc.). Of course, combinations of any of the above should also be included within the scope of computer-readable media.

The electronic control device 900 further includes a system memory 908 with computer storage media in the form of volatile and non-volatile memory, such as ROM and RAM. A basic input/output system (BIOS) with appropriate computer-based routines assists in transferring information between components within the electronic control device 900 and is normally stored in ROM. The RAM portion of the system memory 908 typically contains data and program modules that are immediately accessible to or presently being operated on by a processor 904, e.g., an operating system, application programming interfaces, application programs, program modules, program data, and other instruction-based computer-readable codes.

With continued reference to FIG. 6, the electronic control device 900 may also include other removable or non-removable, volatile or non-volatile, transitory or non-transitory computer storage media products. For example, the electronic control device 900 may include a non-removable memory interface 910 that communicates with and controls a hard disk drive 912, e.g., a non-removable, non-volatile magnetic medium; and a removable, non-volatile memory interface 914 that communicates with and controls a magnetic disk drive unit 916 (which reads from and writes to a removable, non-volatile magnetic disk 918), an optical disk drive unit 920 (which reads from and writes to a removable, non-volatile optical disk 922, such as a CD ROM), a Universal Serial Bus (USB) port 921 for use in connection with a removable memory card, etc. However, it is envisioned that other removable or non-removable, volatile or non-volatile computer storage media can be used in an exemplary computing system environment 902, including, but not limited to, magnetic tape cassettes, DVDs, digital video tape, solid state RAM, solid state ROM, etc. These various removable or non-removable, volatile or non-volatile magnetic media are in communication with the processor 904 and other components of the electronic control device 900 via a system bus 906. The drives and their associated computer storage media, discussed above and illustrated in FIG. 6, provide storage of operating systems, computer-readable instructions, application programs, data structures, program modules, program data, and other instruction-based, computer-readable code for the electronic control device 900 (whether duplicative or not of this information and data in the system memory 908).

A user may enter commands, information, and data into the electronic control device 900 through certain attachable or operable input devices, such as the user interface 124 shown in FIG. 1, via a user input interface 928. A variety of such input devices may be utilized, e.g., a microphone, a trackball, a joystick, a touchpad, a touch-screen, a scanner, etc., including any arrangement that facilitates the input of data and information to the electronic control device 900 from an outside source. As discussed, these and other input devices are often connected to the processor 904 through the user input interface 928 coupled to the system bus 906, but may be connected by other interface and bus structures, such as a parallel port, game port, or a USB. Still further, data and information can be presented or provided to a user in an intelligible form or format through certain output devices, such as a monitor 930 (to visually display this information and data in electronic form), a printer 932 (to physically display this information and data in print form), a speaker 934 (to audibly present this information and data in audible form), etc. All of these devices are in communication with the electronic control device 900 through an output interface 936 coupled to the system bus 906. It is envisioned that any such peripheral output devices be used to provide information and data to the user.

The electronic control device 900 may operate in a network environment 938 through the use of a communications device 940, which is integral to the electronic control device 900 or remote therefrom. This communications device 940 is operable by and in communication with the other components of the electronic control device 900 through a communications interface 942. Using such an arrangement, the electronic control device 900 may connect with or otherwise communicate with one or more remote computers, such as a remote computer 944, which may be a personal computer, a server, a router, a network personal computer, a peer device, or other common network nodes, and typically includes many or all of the components described above in connection with the electronic control device 900. Using appropriate communication devices 940, e.g., a modem, a network interface or adapter, etc., the computer 944 may operate within and communicate through a local area network (LAN) and a wide area network (WAN), but may also include other networks such as a virtual private network (VPN), an office network, an enterprise network, an intranet, the Internet, etc.

As used herein, the electronic control device 900 includes or is operable to execute appropriate custom-designed or conventional software to perform and implement the processing steps of the method and system of the present disclosure, thereby forming a specialized and particular computing system. Accordingly, the method and system may include one or more electronic control devices 900 or similar computing devices having a computer-readable storage medium capable of storing computer-readable program code or instructions that cause the processor 904 to execute, configure, or otherwise implement the methods, processes, and transformational data manipulations discussed hereinafter in connection with the present disclosure. Still further, the electronic control device 900 may be in the form of a personal computer, a personal digital assistant, a portable computer, a laptop, a palmtop, a mobile device, a mobile telephone, a server, or any other type of computing device having the necessary processing hardware to appropriately process data to effectively implement the fluid injector system, the computer program product and the computer-implemented method of the present disclosure.

It will be apparent to one skilled in the relevant arts that the system may utilize databases physically located on one or more computers which may or may not be the same as their respective servers. For example, programming software on the electronic control device 900 can control a database physically stored on a separate processor of the network or otherwise.

In some examples, the electronic control device 900 may be programmed so that automatic refill occurs based upon a preprogrammed trigger minimum volume in the respective syringes 132. For example, when the volume of fluid remaining in at least one of the syringes 132 is less than a programmed volume, a syringe refill procedure is automatically initiated by the electronic control device 900. The electronic control device 900 associated with the fluid injector system 100 may determine that the preprogrammed trigger minimum volume has been reached by tracking the fluid volume dispensed from the respective syringes 132 during operation of the fluid injector system 100. Alternatively, fluid level sensors may be incorporated into the fluid injector system 100 and inputs from these fluid level sensors may be provided to the electronic control device 900 so that the electronic control device 900 may determine when the preprogrammed trigger minimum volume has been reached in at least one of the syringes 132. The fill volume and rate of refill can be preprogrammed in the electronic control device 900. The automatic refill procedure can be stopped either automatically by the electronic control device 900 or may be manually interrupted. In addition, an automatic refill procedure may be initiated when, at the completion of a fluid injection procedure, there is not enough fluid in at least one of the syringes 132 to perform the next programmed fluid injection procedure.

During a refill procedure it is possible that one or more of the bulk fluid sources 120 associated with the respective syringes 132 may become empty (e.g., initially lack sufficient fluid to complete a full refill of the one or more syringes 132). A replacement bulk fluid source 120 is, therefore, necessary and replacement of such bulk fluid source 120 is desirably made quickly. The fluid injector system 100 may have an indicator, such as an audible and/or visual indicator, to indicate to the operator that a change of the bulk fluid source 120 is necessary before the fluid injector system 100 may be used.

As described above, the fluid injector system 100 may automatically or manually prime the MUDS 130 and the SUDS 190 once the SUDS 190 is securely connected to the MUDS 130, for example, as sensed by the sensor 242. During such a priming operation, saline, or another suitable diluent, is injected from the MUDS 130 through the connection port 192, into the tubing 208 of the SUDS 190, and into the waste reservoir 156. Flow of the priming fluid toward the waste reservoir 156 purges extant fluid from the fluid injector system 100 by forcing any extant fluid in SUDS 190 and/or in the manifold 148 of the MUDS 130 out the distal end 212 of the tubing 208. The priming fluid thus replaces any extant fluid in the SUDS 190 with the priming fluid. During the priming operation, various components of the fluid injector system 100 may communicate with the electronic control device 900 to continuously or intermittently monitor a pressure generated during delivery of the priming fluid from the MUDS 130 through the SUDS 190. By monitoring this pressure, the electronic control device 900 can determine various characteristics of the SUDS 190 and associated components. In various aspects or examples of the present disclosure, the electronic control device 900 may be utilized to determine whether the SUDS 190 has been previously used, whether the SUDS 190 has been fully primed, the presence of an additional fluid path set component connected to the connector 214 of the SUDS 190, the length of the SUDS 190, and/or the age of the SUDS 190. These and other aspects and examples of the present disclosure will be discussed in detail herein.

In some aspects or examples, the electronic control device 900 may be utilized to determine whether the SUDS 190 has been previously used based on an extant fluid displaced from the SUDS 190 during the priming thereof. If unused, the SUDS 190 may be initially filled with a gas, such as air or another gas injected into the SUDS 190 during manufacture and/or packaging, whereas a used SUDS 190 may be filled with a liquid, such as a residual medical liquid from a previously performed injection protocol. The electronic control device 900 may determine whether the extant fluid displaced from the SUDS 190 during priming was a gas, indicating that the SUDS 190 is unused, or a liquid, indicating that the SUDS 190 was previously used. The determination of whether the extant, displaced fluid is a gas or liquid may be based on a pressure profile generated during priming of the SUDS 190. The pressure profile may be obtained by measuring the pressure generated as a result of displacing extant fluid from the SUDS 190 at predetermined time intervals as the priming fluid is injected through the SUDS 190 during the priming operation of the SUDS 190. Hereinafter, this pressure profile, which represents actual measured pressure of the priming operation over time, will be referred to as the “distinct pressure profile”.

To determine whether the extant fluid displaced from the SUDS 190 during priming was a gas or a liquid, the electronic control device 900 may compare the distinct pressure profile to a predetermined pressure profile. The predetermined pressure profile is representative of pressure expected to be generated within an exemplary SUDS by the priming fluid over a course of a priming operation performed on the exemplary SUDS. In particular, the predetermined pressure profile may be representative of the pressure expected to be generated in an unused SUDS during an identical priming operation to that performed on the actual subject SUDS 190. The predetermined pressure profile may be obtained through pressure measurement of an exemplary SUDS known to be unused.

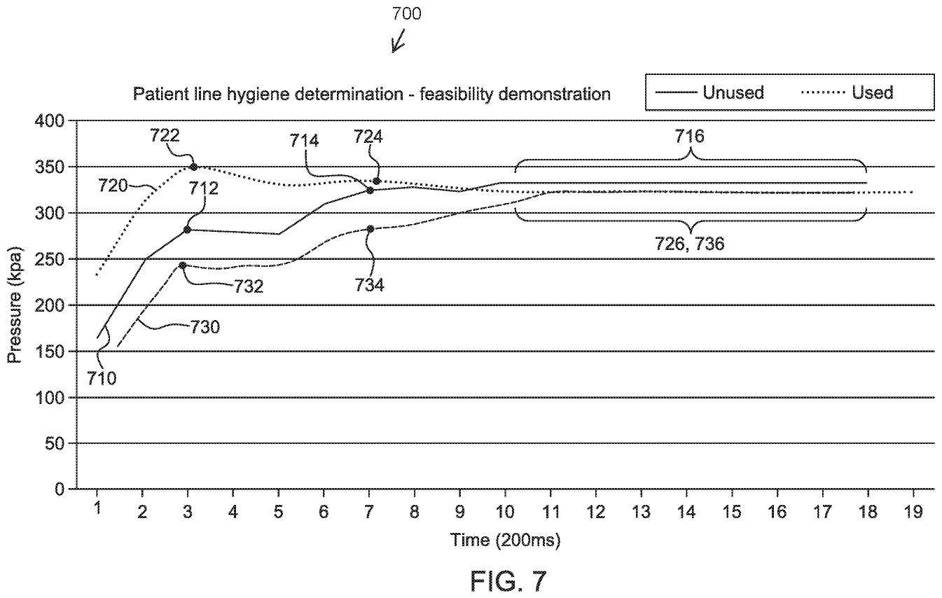

Correlation between the distinct pressure profile and the predetermined pressure profile is indicative of whether the SUDS 190, prior to the priming operation, contained gas as the extant fluid or liquid as the extant fluid. Predetermined pressure profiles for various exemplary SUDS may be presented graphically to facilitate interpretation and comparison of a predetermined pressure profile and a distinct pressure profile. FIG. 7 illustrates a graph 700, including a graphical representation of a first predetermined pressure profile 710 and a graphical representation of a second predetermined pressure profile 720, with time on the x-axis and pressure on the y-axis. In the example shown in FIG. 7, time is presented in units of 200 milliseconds (ms) and pressure is presented in units of kilopascals (kPa). For the first predetermined pressure profile 710, the graphed pressure corresponds to expected pressure for an exemplary SUDS known to be unused, whereas for the second predetermined pressure profile 720, the graphed pressure corresponds to expected pressure for an exemplary SUDS known to have been used. FIG. 7 further includes a graphical representation of a distinct pressure profile 730 generated during priming of the SUDS 190. The graphed time begins at 0 ms, corresponding to initiation of the priming operation, and extends through completion of the priming operation.

Various events over the course of the priming operation may be appreciated from the graph 700 of FIG. 7 by identifying specific pressure values and/or changes in pressure values over time. A first inflection point 712 of the first predetermined pressure profile 710 may correspond to a time at which the priming fluid passes through the one-way check valve 236 of the fluid inlet port 202 of the exemplary SUDS. In particular, the pressure change at a first inflection point 712 indicates the one-way check valve 236 being opened in response to an accumulation of fluid pressure in the MUDS 130 leading into the exemplary SUDS. Similarly, a second inflection point 714 of the predetermined pressure profile 710 may correspond to a time at which the priming fluid passes through the one-way check valve 280 in the connector 214 at the distal end of the exemplary SUDS. The pressure change at the second inflection point 714 indicates the one-way check valve 280 being opened in response to an accumulation of fluid pressure in the exemplary SUDS. Subsequent to the second inflection point 714, pressure fluctuation of the predetermined pressure profile 710 settles to a steady state portion 716 corresponding to a time interval over which the priming fluid flows freely through both of the one way check valves 236, 280 of the exemplary SUDS.

With continued reference to FIG. 7, the graphical representation of the second predetermined pressure profile 720 may include a first inflection point 722, a second inflection point 724, and a steady state portion 726. The first inflection point 722 may correspond to a time at which the priming fluid passes through the one-way check valve 236 of the fluid inlet port 202 of the exemplary SUDS, the second inflection point 724 may correspond to a time at which the priming fluid passes through the one-way check valve 280 in the connector 214 of the exemplary SUDS, and the steady state portion 726 may correspond to a time interval over which the priming fluid flows freely through both of the one way check valves 236, 280 of the exemplary SUDS.

With continued reference to FIG. 7, the graphical representation of the distinct pressure profile 730 may include a first inflection point 732, a second inflection point 734, and a steady state portion 736. The first inflection point 732 may correspond to a time at which the priming fluid passes through the one-way check valve 236 of the fluid inlet port 202 of the SUDS 190, the second inflection point 734 may correspond to a time at which the priming fluid passes through the one-way check valve 280 in the connector 214 of the SUDS 190, and the steady state portion 736 may correspond to a time interval over which the priming fluid flows freely through both of the one way check valves 236, 280 of the SUDS 190.

In FIG. 7, the exemplary SUDS represented by the first predetermined pressure profile 710 is unused, meaning that the extant fluid contained in the exemplary SUDS prior to priming was a gas. In contrast, the exemplary SUDS represented by the second predetermined pressure profile 720 was previously used, meaning that the extant fluid contained in the exemplary SUDS prior to priming was at least partly a liquid. The distinct pressure profile 730 is illustrative of an unused SUDS 190.

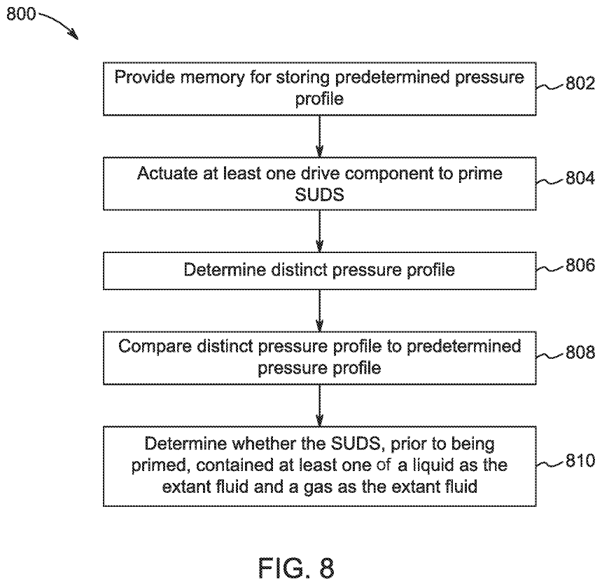

Having generally described the characteristics of the pressure profiles in general, a method 800 of detecting reuse of the SUDS 190 in accordance with some aspects and examples of the present disclosure will be described with reference to FIG. 8. At step 802, the method 800 may include providing a memory for storing the predetermined pressure profile 710. The memory may be, for example, the hard disk drive 912 or another memory device integral with or in communication with the electronic control device 900. In particular, the memory may store, as a database, the individual pressure measurements and corresponding time indices of the first and second predetermined pressure profiles 710, 720.

With continued reference to FIG. 8, at step 804, the method 800 may further include actuating at least one drive component, such as one or more of the piston elements 103, of the fluid injector system 100 to prime the SUDS 190. The priming operation of step 804 may be performed substantially as described above, including the injection of saline, or another suitable diluent, from the MUDS 130 through the connection port 192, into the tubing 208 of the SUDS 190, and into the waste reservoir 156.

With continued reference to FIG. 8, at step 806, the method 800 may further include determining the distinct pressure profile 730, as described above, by measuring the pressure generated during the priming of the SUDS 190 during the priming operation of step 804. In some aspects or examples, the pressures generated during the priming of the SUDS 190 represented in the distinct pressure profile 730 may be obtained and/or derived by measuring the current drawn by the electro-mechanical drive component, i.e. the motor current, of the piston element 103 injecting the priming fluid. In other aspects or examples, the pressures generated during the priming of the SUDS 190 represented in the distinct pressure profile 730 may be obtained and/or derived by one or more pressure transducers (not shown) mounted in the MUDS 130 and/or the SUDS 190 in fluid communication with the priming fluid. Other methods of pressure measurement may be appreciated by those of ordinary skill in the art. In some aspects or examples, each pressure measurement of the distinct pressure profile 730, along with a corresponding time index indicating the relative time at which each pressure measurement occurred, may be stored in the hard disk drive 912 or another memory device integral with or in communication with the electronic control device 900.

With continued reference to FIG. 8, at step 808, the method 800 may further include comparing the distinct pressure profile 730 to at least one of the first and second predetermined pressure profiles 710, 720. Various methods of comparing the distinct pressure profile 730 to at least one of the first and second predetermined pressure profiles 710, 720 may be utilized. In some aspects or examples, the electronic control device 900 may compare the pressure measurements and/or time indices associated with the first inflection point 712, the second inflection point 714, and the steady state portion 716 of the first predetermined pressure profile 710 with the pressure measurements and/or time indices associated with the first inflection point 732, the second inflection point 734, and the steady state portion 736 of the distinct pressure profile 730. Alternatively, or in addition, the electronic control device 900 may compare the pressure measurements and/or time indices associated with the first inflection point 722, the second inflection point 724, and the steady state portion 726 of the second predetermined pressure profile 720 with the pressure measurements and/or time indices associated with the first inflection point 732, the second inflection point 734, and the steady state portion 736 of the distinct pressure profile 730. In some aspects or examples, the electronic control device 900 may compare specific pressure measurements at similar or identical time indices of the first and/or second predetermined pressure profiles 710, 720 and the distinct pressure profile 730.

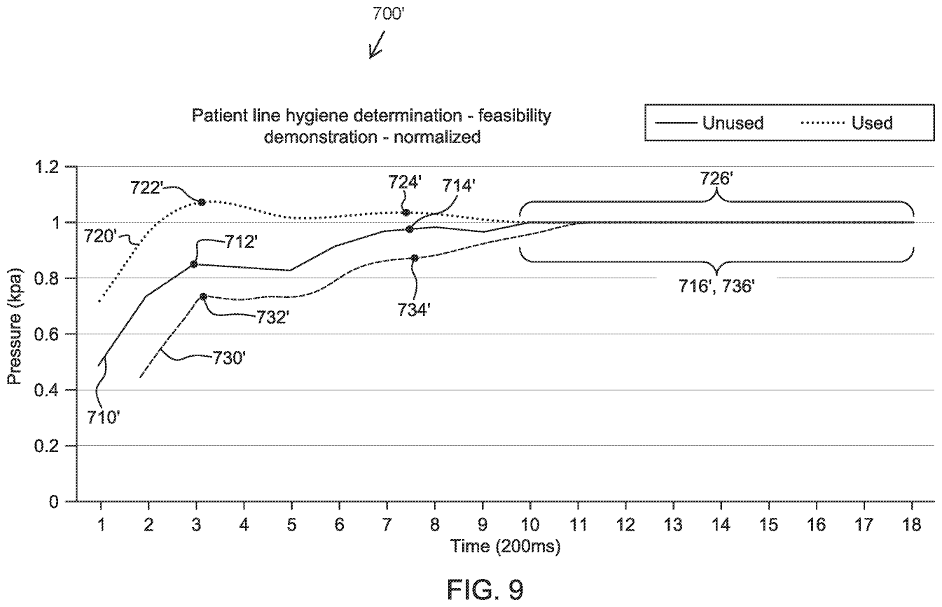

In some aspects or examples, step 808 may include normalizing, with the electronic control device 900, one or both of the first and/or second predetermined pressure profiles 710, 720 and the distinct pressure profile 730 to facilitate comparison of the first and/or second predetermined pressure profile(s) 710, 720 and the distinct pressure profile 730. In particular, the distinct pressure profile 730 may be normalized about the steady state portion 736 such that the pressure values within the steady state portion 736 are normalized to a value of one (1). The normalization may be performed by dividing each individual pressure measurement of the distinct pressure profile 730 by the steady state pressure value (e.g., an average of the values within the steady state portion 736). The first and/or second predetermined pressure profile(s) 710, 720 may be normalized about the steady state portion 716 thereof in the same manner. FIG. 9 shows a normalized graph 700′ of the graph of FIG. 7, with normalized first and second predetermined pressure profiles 710′, 720′ and a normalized distinct pressure profile 730′ being normalizations of the first and second predetermined pressure profiles 710, 720 and the distinct pressure profile 730 of FIG. 7. Normalizing the first and/or second predetermined pressure profile(s) 710, 720 and the distinct pressure profile 730 facilitates comparison regardless of individual machine and/or the calibration differences of the fluid injector system 100 as the steady state portions 716′, 726′, 736′ are normalized about a value of one (1). With the first and/or second predetermined pressure profile(s) 710, 720 and the distinct pressure profile 730 normalized, the electronic control device 900 may, in some aspects or examples, compare an area under a curve of the normalized first and/or second predetermined pressure profile(s) 710′, 720′ to an area under a curve of the normalized distinct pressure profile 730′.

In some aspects or examples, step 808 may include comparing linear trendlines of the first and/or second predetermined pressure profile(s) 710, 720 and the distinct pressure profile 730 over a predefined duration. A portion of the predetermined pressure profile 730 between two predetermined time indices may be fitted with a best-fit straight line. A portion of the distinct pressure profile 730 between the same two time indices may similarly be fitted with a best-fit straight line. The best-fit straight lines of the first and/or second predetermined pressure profile(s) 710, 720 and the distinct pressure profile 730 may then be compared to determine characteristics of the SUDS 190 during the priming operation.

In other aspects or examples, step 808 may include a plurality of the comparison methods discussed above. Each of the comparison methods may be weighted as part of an overall comparison score from which conclusions regarding the first and/or second predetermined pressure profile(s) 710, 720 and the distinct pressure profile 730 may be drawn.

With continued reference to FIG. 8, at step 810, the method 800 may further include determining, based on at least one result of the comparison of step 808, whether the SUDS 190, prior to the priming thereof at step 804, contained at least one of a liquid as the extant fluid and a gas as the extant fluid. Various methods may be used to make the determination at step 810. In general, the electronic control device 900 may determine a correlation between one or more features and/or values of the first and/or second predetermined pressure profile(s) 710, 720 and the distinct pressure profile 730 compared at step 808. If the correlation is within a specified tolerance, the electronic control device 900 may determine that the SUDS 190, prior to being primed, contained an extant fluid of at least partly the same phase (e.g. liquid or gas) as the extant fluid contained in the exemplary SUDS.

In some aspects or examples, the electronic control device 900 may determine that the SUDS 190 contained a liquid or a gas as the extant fluid based on a correlation within a specified tolerance of the first inflection points 712, 722, 732 the second inflection points 714, 724, 734 and/or the steady state portions 716, 726, 736 of the first and/or second predetermined pressure profile 710, 720 and the distinct pressure profile 730. In the example shown in FIG. 7, the first inflection point 712 of the first predetermined pressure profile 710 occurs at approximately 275 kpa and 700 ms, whereas the first inflection point 722 of the second predetermined pressure profile 720 occurs at approximately 350 kpa and 750 ms. FIG. 7 further shows that the first inflection point 732 of the distinct pressure profile 730 occurs at approximately 240 kpa and 650 ms. If the correlation (e.g., the difference between the pressures and/or the difference between the time indices) falls within a specified tolerance (e.g., 10% difference), the electronic control unit 900 may determine that the SUDS 190 contained extant fluid in the same phase (e.g. liquid or gas), prior to the priming operation, as the exemplary SUDS. In contrast, if the correlation falls outside the specified tolerance, the electronic control unit 900 may determine that the SUDS 190 contained extant fluid in a different phase, prior to the priming operation, than the exemplary SUDS. In this example, the correlation of the pressures and time indices of the first predetermined pressure profile 710 and the distinct pressure profile 730, and/or the correlation of the pressures and time indices of the second predetermined pressure profile 720 and the distinct pressure profile 730 fall outside the specified tolerance, and therefore the electronic control unit 900 may determine that the SUDS 190 contained extant fluid in a different phase, prior to the priming operation, than the exemplary SUDS. As the exemplary SUDS contained air prior to the priming operation, the electronic control unit 900 may therefore determine that the SUDS 190 contained liquid prior to being primed and thus had been used in a previously performed injection protocol.

In some aspects or examples, step 810 may include determining that the SUDS 190 contained a liquid or a gas as the extant fluid based on a correlation of the normalized first and/or second predetermined pressure profile(s) 710′, 720′ and the normalized distinct pressure profile 730′. In particular, the electronic control device 900 may determine whether the correlation between the area under the curve of the normalized distinct pressure profile 730′ is within a specified tolerance to the area under the curve of the first and/or second predetermined pressure profile(s) 710′, 720′. If the electronic control device 900 makes a determination in the affirmative, the extant fluid contained in the SUDS 190 prior to priming was in the same phase as the extant fluid in the exemplary SUDS. If the electronic control device 900 makes a determination in the negative, the extant fluid contained in the SUDS 190 prior to priming was in a different phase than the extant fluid in the exemplary SUDS. Based on this determination, the electronic control unit 900 may determine whether the SUDS 190 has been used in a previously performed injection protocol.

In some aspects or examples, step 810 may include determining that the SUDS 190 contained a liquid or a gas as the extant fluid based on a correlation of best-fit lines of the first and second predetermined pressure profiles 710, 720 and the distinct pressure profile 730. As with the above-described aspects, the electronic control unit 900 may determine that the SUDS 190 contained extant fluid in the same phase, prior to the priming operation, as the exemplary SUDS if the correlation falls within a specified tolerance. Conversely, the electronic control unit 900 may determine that the SUDS 190 contained extant fluid in a different phase, prior to the priming operation, than the exemplary SUDS if the correlation falls outside the specified tolerance. Based on this determination, the electronic control unit 900 may determine whether the SUDS 190 has been used in a previously performed injection protocol.

In other aspects or examples, step 810 may include a plurality of the above-described methods for determining whether the extant fluid contained in the SUDS 190, prior to being primed, was a liquid or a gas. Each of these determinations may be weighted as part of an overall score from which conclusions regarding previous usage of the SUDS 190 may be drawn.

In some aspects or examples, the determination made at step 810 may be used to generate an alert indicating whether the SUDS 190 had been previously used. In particular, the electronic control unit 900 may generate the alert in response to determining that the SUDS 190, prior to being primed, contained at least part liquid as the extant fluid and, therefore, was used in a previously performed injection protocol. The alert may be generated by the electronic control unit 900 in the form a visual, audio, tactile, or other sensory output configured to prompt the attention of a physician or other care provider. In some aspects or examples, the alert may be a graphic displayed on one or more user interfaces 124 of the fluid injector system 100, a noise emitted from the speaker 934 of the fluid injector system 100, or a combination thereof.

In some aspects or examples, the determination made at step 810 may be input into a compliance report generated by the electronic control unit 900. The compliance report may be displayed on one or more user interfaces 124 of the fluid injector system 100 to provide visual feedback about compliance with hygienic practices, e.g., routine replacement of the SUDS 190. The compliance report may also be stored in a compliance database for future analysis of hygienic practices. Further details of generating, displaying, and analyzing a compliance report utilizing the fluid injector system 100 are provided in International Patent Application No. PCT/US2019/026659, filed on Apr. 9, 2019 and entitled “System and Methods for Monitoring Hygiene Practices Associated with Use of Power Fluid Injector Systems”, the disclosure of which is hereby incorporated by reference in its entirety.

In some aspects or examples, the determination made at step 810 may prevent commencement of the injection protocol if the electronic control unit 900 determines that the SUDS 190 had been previously used. In particular, the electronic control unit 900 may prohibit commencement of an enabled injection protocol in response to determining that the SUDS 190, prior to being primed, contained at least part liquid as the extant fluid and, therefore, was used in a previously performed injection protocol. Conversely, the electronic control unit 900 may permit commencement of the enabled injection protocol in response to determining that the SUDS 190, prior to being primed, contained gas as the extant fluid and, therefore, is unused.

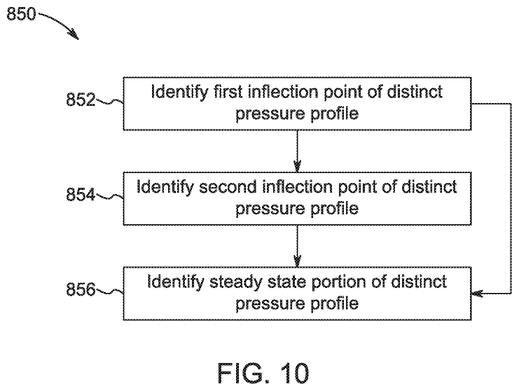

As noted above, monitoring the pressure generated during the priming operation may also be utilized to determine characteristics of the SUDS 190 other than the extant fluid contained therein. In some aspects or examples, the electronic control unit 900 may determine whether the SUDS 190 has been fully primed based on the distinct pressure profile 730. FIG. 10 shows a method 850 that may be used in determining whether the SUDS 190 has been fully primed. At step 852, the method 850 may include identifying the first inflection point 732 in the distinct pressure profile 730 caused by the at least one fluid having passed through the one-way check valve 236 of the SUDS 190. The electronic control unit 900 may identify the first inflection point 732 by identifying a sharp rise in pressure followed by a plateau in pressure due to opening of the check valve 236. In some examples or aspects, the electronic control unit 900 may compare the distinct pressure profile 730 to the first and/or second predetermined pressure profile(s) 710, 720 and determine whether the first inflection point 732 of the distinct pressure profile 730 correlates within a specified tolerance to the first inflection point 712 of the first predetermined pressure profile 710 and/or the first inflection point 722 of the second predetermined pressure profile 720.

With continued reference to FIG. 10, the method 850 may further include, at step 854, identifying the second inflection point 734 in the distinct pressure profile 730 caused by the at least one fluid having passed through the one-way check valve 280 of the SUDS 190. The electronic control unit 900 may identify the second inflection point 734 by comparing the distinct pressure profile 730 to the first and/or second predetermined pressure profile(s) 710, 720 and determining that the second inflection point 734 of the distinct pressure profile 730 correlates within a specified tolerance to the second inflection point 714, 724 of the first and/or second predetermined pressure profile(s) 710, 720. If the electronic control unit 900 cannot identify the second inflection point 734 of the distinct pressure profile 730 correlated within the specified tolerance, the electronic control unit 900 may determine that the priming fluid has not reached the one-way check valve 280 and, thus, the SUDS 190 has not been fully primed.

With continued reference to FIG. 10, the method 850 may further include, at step 856, identifying the steady state portion 736 in the distinct pressure profile 730 caused by the priming fluid flowing freely after having passed through both one-way check valves 236, 280 of the SUDS 190. The electronic control unit 900 may identify the steady state portion 736 by determining that the pressure is constant after a predetermined period of time during the priming operation. The electronic control unit 900 may identify the steady state portion 736 by comparing the distinct pressure profile 730 to the first and/or second predetermined pressure profile(s) 710, 720 and determining that the steady state portion 736 of the distinct pressure profile 730 correlates within a specified tolerance to the steady state portion 716, 726 of the first and/or second predetermined pressure profile(s) 710, 720. If the electronic control unit 900 cannot identify the steady state portion 736 of the distinct pressure profile 730 as being so correlated within the specified tolerance, the electronic control unit 900 may determine that the priming fluid has not reached steady state and, thus, the SUDS 190 has not been fully primed. If the electronic control unit 900 identifies either the second inflection point 734 at step 854 or the steady state portion 736 at step 856, the electronic control unit 900 may determine that the SUDS has been fully primed.

In some aspects or examples of the method 850, only one of steps 854 and 856 may be performed. It is also noted that the method 850 as described above presumes that the SUDS 190 includes both one-way check valves 236 and 280. However, in some aspects or examples, the one-way check valve 236 may be omitted, and the method 850 may be performed without step 852 since the first inflection point 732 would not be exhibited in the absence of the one-way check valve 236.

In some aspects or examples, the electronic control unit 900 may determine a length of the SUDS 190 based on the distinct pressure profile 730. The electronic control unit 900 may determine the elapsed time between the first inflection point 732 and the second inflection point 734 of the distinct pressure profile 730, indicative of the time between the openings of the one-way check valves 236, 280. The electronic control unit 900 may further determine the volume of fluid injected into the SUDS 190, based on, for example, displacement of the piston elements 103 during this elapsed time, to determine an internal volume of the SUDS 190. The internal volume of the SUDS 190 can be converted to length by dividing the internal volume by a known cross-sectional area of the tubing 208.

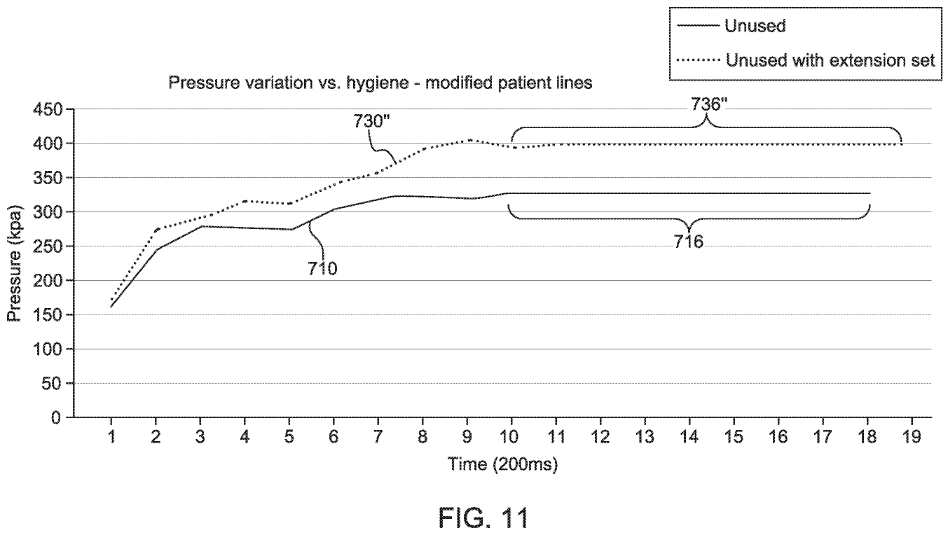

In some aspects or examples, the electronic control unit 900 may determine a presence or absence of an additional fluid path set component connected to the connector 214 of the SUDS 190 based on the distinct pressure profile 730. The presence of the additional fluid path set component, such as an extension line, may introduce additional characteristics to the distinct pressure profile 730, such as a third inflection point or an increased steady state pressure. In particular, the additional fluid path set component may introduce an additional restriction to the fluid path, such as an additional one-way check valve or a narrower lumen, that causes a pressure inflection or increased steady state pressure as the priming fluid passes therethrough. FIG. 11 shows a graph of the first predetermined pressure profile 710 and a modified distinct pressure profile 730″ representative of the SUDS 190 with the additional fluid path set component attached. The electronic control unit 900 may identify the increased pressure of the steady state portion 736″ of the modified distinct pressure profile 730″ relative to the pressure of the steady state portion 716 of the first predetermined pressure profile to determine that the additional fluid path set component is connected to the SUDS 190.

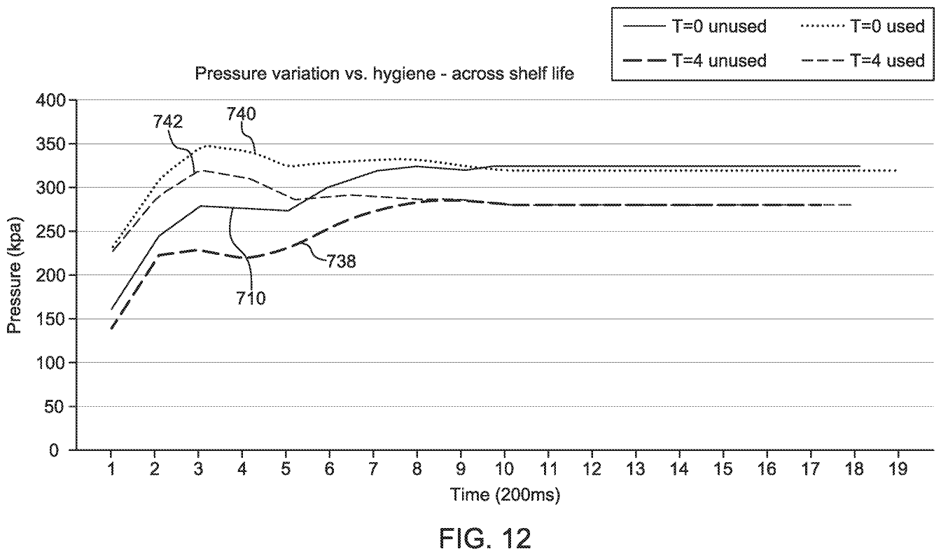

In some aspects or examples, the electronic control unit 900 may account for any differences in pressure profiles depending on an age of the SUDS 190. FIG. 12 shows the first predetermined pressure profile 710, representative of the relatively new or pristine (i.e. not aged) exemplary SUDS, in comparison to an aged second predetermined pressure profile 738 representative of an unused but aged exemplary SUDS. Additionally, a third predetermined pressure profile 740 is representative of a relatively new and previously-used exemplary SUDS, and a fourth predetermined pressure profile 742 is representative of an aged and previously-used exemplary SUDS. Various characteristics of the pressure profiles 710, 738, 740, 742 such as inflection points and steady state portions thereof may be identified, compared, and analyzed by the electronic control unit 900 as generally described herein in steps 808 and 810 of the method 800 to determine the age of the SUDSs represented by the pressure profiles 710, 738, 740, 742.

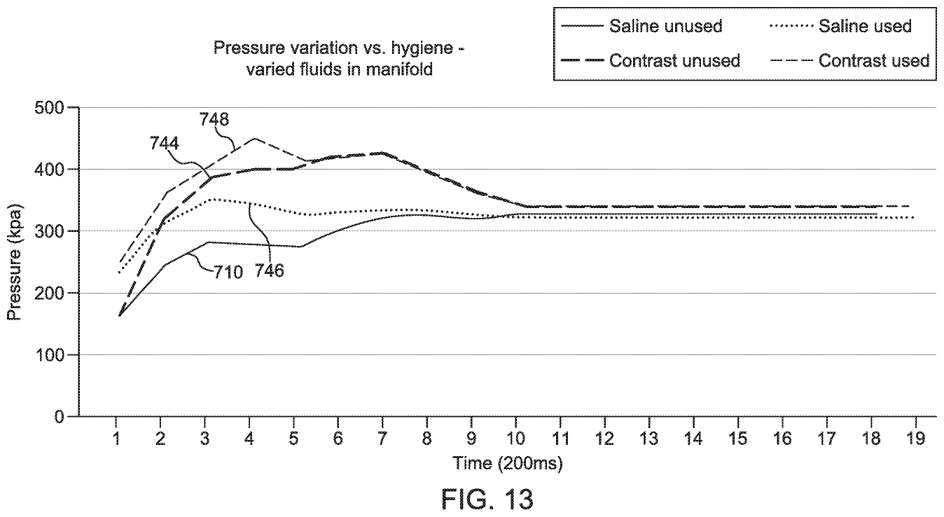

In some aspects or examples, the electronic control unit 900 may account for any differences in pressure profiles depending on what fluid is in the manifold 148 at the start of the priming operation. FIG. 13 illustrates the first predetermined pressure profile 710 representative of the unused exemplary SUDS coupled with the presence of saline as the fluid in the manifold 148. Also illustrated are predetermined pressure profiles 744, 746, 748 representative of (i) an unused exemplary SUDS with contrast as the fluid in the manifold 148, (ii) a previously used exemplary SUDS with saline as the fluid in the manifold 148, and (iii) a previously used exemplary SUDS with contrast as the fluid in the manifold 148, respectively. Various characteristics of the predetermined pressure profiles 710, 744, 746, 748, such as inflection points and steady state portions thereof, may be identified, compared, and analyzed by the electronic control unit 900 as generally described herein in steps 808 and 810 of the method 800. In this manner, the type of fluid (e.g., saline or contrast) in the manifold 148 at the start of the priming operation can be accounted for during comparison between the distinct pressure profile and the predetermined pressure profile.

In some aspects or examples of the present disclosure, the methods 800, 850, as well as the other methods and processes described herein, may be implemented in the fluid injector system 100 by a computer program product. The computer program product may include at least one non-transitory computer-readable medium having one or more instructions executable by at least one processor to cause the at least one processor to execute all or part of the method 800. In some examples or aspects, the at least one non-transitory computer-readable medium and the at least one processor may include or correspond to the memory 908 and processor 904, respectively, as described above with reference to FIG. 6.

While several examples of fluid injector systems, computer program products, and associated methods are shown in the accompanying drawings and described hereinabove in detail, other examples will be apparent to, and readily made by, those skilled in the art without departing from the scope and spirit of the disclosure. For example, it is to be understood that this disclosure contemplates that, to the extent possible, one or more features of any example can be combined with one or more features of any other example. Accordingly, the foregoing description is intended to be illustrative rather than restrictive.

We claim:

1. A fluid injector system configured to perform an injection protocol in connection with a diagnostic imaging procedure, the fluid injector system comprising:
a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line;
a control device operatively associated with at least one drive component configured to pressurize and inject at least one fluid through a subject administration line into a patient, the control device including at least one processor programmed or configured to perform an operation comprising:
actuating the at least one drive component to prime the subject administration line with the at least one fluid as the priming fluid;
determining a distinct pressure profile indicative of a measurement of current pressure generated during the priming of the subject administration line with the at least one fluid over the course of the priming operation performed therewith;
comparing the distinct pressure profile to the predetermined pressure profile; and
determining, based on a result of the comparison, whether the subject administration line, prior to the priming thereof, contained therein at least one of a liquid as the extant fluid and a gas as the extant fluid.

2. The fluid injector system of claim 1, wherein the exemplary administration line is one of:
(i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and
(ii) a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation.

3. The fluid injector system of claim 2, wherein the exemplary administration line is the unused administration line and upon the comparison resulting in a correlation within a specified tolerance between the distinct pressure profile and the predetermined pressure profile, the at least one processor is configured to determine that the subject administration line contained the gas as the extant fluid.

4. The fluid injector system of claim 3, wherein the operation further comprises:
upon the comparison resulting in the correlation within the specified tolerance between the distinct pressure profile and the predetermined pressure profile, generating an alert indicating that the subject administration line had not been used prior to being primed with the at least one fluid during the priming operation.

5. The fluid injection system of claim 3, wherein the operation further comprises:
upon the comparison resulting in the correlation within the specified tolerance between the distinct pressure profile and the predetermined pressure profile, permitting performance of the injection protocol.

6. The fluid injector system of claim 3, wherein the subject administration line, like the exemplary administration line, comprises at least one check valve precluding fluid flow in a proximal direction; and
wherein the comparison resulting in the correlation within the specified tolerance between the distinct pressure profile and the predetermined pressure profile comprises at least one of:
identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line;
normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and
normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

7. The fluid injector system of claim 2, wherein the exemplary administration line is the previously used administration line and upon the comparison resulting in a correlation within a specified tolerance between the distinct pressure profile and the predetermined pressure profile, the at least one processor is configured to determine that the subject administration line contained the liquid as the extant fluid.

8. The fluid injector system of claim 7, wherein the operation further comprises:
upon the comparison resulting in the correlation within the specified tolerance between the distinct pressure profile and the predetermined pressure profile, generating an alert indicating that the subject administration line had been used prior to being primed with the at least one fluid during the priming operation.

9. The fluid injector system of claim 7, wherein the subject administration line, like the exemplary administration line, comprises at least one check valve precluding fluid flow in a proximal direction; and
wherein the comparison resulting in the correlation within the specified tolerance between the distinct pressure profile and the predetermined pressure profile comprises at least one of:
identifying at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line;
normalizing the distinct pressure profile about a steady state value thereof, normalizing the predetermined pressure profile about a steady state value thereof, and determining that an area under a curve of the normalized distinct pressure profile correlates to an area under a curve of the predetermined pressure profile; and
normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

10. The fluid injector system of claim 1, wherein the at least one fluid used in the priming of the subject administration line comprises at least one of (i) a diluent, (ii) a contrast medium and (iii) a mixture of the contrast medium and the diluent.

11. The fluid injector system of claim 1, wherein the determining the distinct pressure profile comprises measuring a motor current of the at least one drive component.

12. The fluid injector system of claim 3, wherein the subject administration line, like the exemplary administration line, comprises a first check valve precluding fluid flow in a proximal direction and a second check valve precluding flow in the proximal direction, the second check valve located distally of the first check valve; and
wherein, during the priming of the subject administration line, the operation further comprises:
identifying a first pressure inflection point in the distinct pressure profile caused by the at least one fluid having passed through the first check valve in the subject administration line; and
identifying at least one of:
a second pressure inflection point in the distinct pressure profile caused by the at least one fluid having passed through the second check valve in the subject administration line; and
a steady state portion of the distinct pressure profile over which the pressure generated within the subject administration line remains substantially constant;
whereupon the subject administration line is determined to be fully primed.

13. The fluid injector system of claim 1, wherein the subject administration line, like the exemplary administration line, comprises a single check valve located in a distal end of the subject administration line, the single check valve precluding fluid flow in a proximal direction; and
wherein, during the priming of the subject administration line, the operation further comprises identifying at least one of:
a pressure inflection point in the distinct pressure profile caused by the at least one fluid having passed through the single check valve in the subject administration line; and
a steady state portion of the distinct pressure profile over which the pressure generated within the subject administration line remains substantially constant;
whereupon the subject administration line at a distal end thereof is determined to have a fluid path set component connected thereto.

14. A computer program product for detecting multiple uses of an administration line using a fluid injector system configured to perform an injection protocol in connection with a diagnostic imaging procedure, the computer program product comprising:
non-transitory computer readable media comprising a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line;
the non-transitory computer readable media further comprising one or more instructions that, when executed by at least one processor, cause the at least one processor to perform an operation comprising:
actuating at least one drive component of the fluid injector system to prime a subject administration line with the priming fluid;
determining a distinct pressure profile indicative of a measurement of current pressure generated during the priming of the subject administration line with the at least one fluid over the course of the priming operation performed therewith;
comparing the distinct pressure profile to the predetermined pressure profile; and
determining, based on a result of the comparison, whether the subject administration line, prior to the priming thereof, contained therein at least one of a liquid as the extant fluid and a gas as the extant fluid.

15. The computer program product of claim 14, wherein the exemplary administration line is one of:
(i) an unused administration line and, as a result thereof, the extant fluid therein is the gas and the predetermined pressure profile thereby represents the pressure expected to be generated by the priming fluid during the priming of the unused administration line as the gas therein is completely displaced thereby over the course of the priming operation; and
(ii) a previously used administration line and, as a result thereof, the extant fluid therein is at least partially a liquid and the predetermined pressure profile thereby represents the pressure expected to be generated by the priming fluid during the priming of the previously used administration line as the liquid therein is completely displaced thereby over the course of the priming operation.

16. The computer program product of claim 15, wherein the exemplary administration line is the unused administration line and upon the comparison resulting in a correlation within a specified tolerance between the distinct pressure profile and the predetermined pressure profile, the one or more instructions, when executed by the at least one processor, cause the at least one processor to determine that the subject administration line contained the gas as the extant fluid.

17. The computer program product of claim 16, wherein the one or more instructions, when executed by the at least one processor, cause the at least one processor to perform a further operation comprising:
upon the comparison resulting in the correlation within the specified tolerance between the distinct pressure profile and the predetermined pressure profile, generating an alert indicating that the subject administration line had not been used prior to being primed with the at least one fluid during the priming operation.

18. The computer program product of claim 16, wherein the one or more instructions, when executed by the at least one processor, cause the at least one processor to perform a further operation comprising:
upon the comparison resulting in the correlation within the specified tolerance between the distinct pressure profile and the predetermined pressure profile, permitting performance of the injection protocol.

19. The computer program product of claim 16, wherein the subject administration line, like the exemplary administration line, comprises at least one check valve precluding fluid flow in a proximal direction; and
wherein the comparison resulting in the correlation within the specified tolerance between the distinct pressure profile and the predetermined pressure profile comprises at least one of:
identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line;
normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and
normalizing the distinct pressure profile about a steady state value thereof, normalizing the predetermined pressure profile about a steady state value thereof, and determining that each point along the distinct pressure profile correlates within a specified tolerance to corresponding points on the predetermined pressure profile.

20. The computer program product of claim 15, wherein the exemplary administration line is the previously used administration line and upon the comparison resulting in a correlation within a specified tolerance between the distinct pressure profile and the predetermined pressure profile, the one or more instructions, when executed by the at least one processor, cause the at least one processor to determine that the subject administration line contained the liquid as the extant fluid.

21. The computer program product of claim 20, wherein the one or more instructions, when executed by the at least one processor, cause the at least one processor to perform a further operation comprising:
upon the comparison resulting in the correlation within the specified tolerance between the distinct pressure profile and the predetermined pressure profile, generating an alert indicating that the subject administration line had been used prior to being primed with the at least one fluid during the priming operation.

22. The computer program product of claim 20, wherein the subject administration line, like the exemplary administration line, comprises at least one check valve precluding fluid flow in a proximal direction; and
wherein the comparison resulting in the correlation within the specified tolerance between the distinct pressure profile and the predetermined pressure profile comprises at least one of:
identifying, with the at least one processor, at least one pressure inflection point in the distinct pressure profile caused by the at least one fluid having passed through the at least one check valve in the subject administration line that correlates to at least one pressure inflection point corresponding thereto in the predetermined pressure profile caused by the priming fluid having passed through the at least one check valve in the exemplary administration line;
normalizing, with the at least one processor, the distinct pressure profile about a steady state value thereof; normalizing, with the at least one processor, the predetermined pressure profile about a steady state value thereof; and determining, with the at least one processor, that an area under a curve of the normalized distinct pressure profile correlates to an area under a curve of the predetermined pressure profile; and
normalizing, with the at least one processor, the distinct pressure profile about a steady state value thereof, normalizing, with the at least one processor, the predetermined pressure profile about a steady state value thereof, and determining, with the at least one processor, that each point along the distinct pressure profile correlates within a specified tolerance to corresponding points on the predetermined pressure profile.

23. The computer program product of claim 14, wherein the at least one fluid used in the priming of the subject administration line comprises at least one of (i) a diluent, (ii) a contrast medium and (iii) a mixture of the contrast medium and the diluent.

24. The computer program product of claim 14, wherein determining the distinct pressure profile comprises measuring, with the at least one processor, a motor current of the at least one drive component.

25. The computer program product of claim 16, wherein the subject administration line, like the exemplary administration line, comprises a first check valve precluding fluid flow in a proximal direction and a second check valve precluding flow in the proximal direction, the second check valve located distally of the first check valve; and
wherein, during priming of the subject administration line, the operation further comprises:
identifying a first pressure inflection point in the distinct pressure profile caused by the at least one fluid having passed through the first check valve in the subject administration line; and
identifying at least one of:
a second pressure inflection point in the distinct pressure profile caused by the at least one fluid having passed through the second check valve in the subject administration line; and
a steady state portion of the distinct pressure profile over which the pressure generated within the subject administration line remains substantially constant;
whereupon the subject administration line is determined to be fully primed.

26. The computer program product of claim 14, wherein the subject administration line, like the exemplary administration line, comprises a single check valve located in a distal end of the subject administration line, the single check valve precluding fluid flow in a proximal direction; and
wherein, during the priming of the subject administration line, the operation further comprises identifying at least one of:
a pressure inflection point in the distinct pressure profile caused by the at least one fluid having passed through the single check valve in the subject administration line; and
a steady state portion of the distinct pressure profile over which the pressure generated within the subject administration line remains substantially constant;
whereupon the subject administration line at a distal end thereof is determined to have a fluid path set component connected thereto.

27. A method for detecting multiple uses of an administration line using a fluid injector system configured to perform an injection protocol in connection with a diagnostic imaging procedure, the method comprising:
providing a memory for storing therein a predetermined pressure profile, the predetermined pressure profile being representative of pressure expected to be generated within an exemplary administration line by a priming fluid over a course of a priming operation performed on the exemplary administration line during which the priming fluid completely displaces an extant fluid from the exemplary administration line;
actuating at least one drive component of the fluid injector system to prime a subject administration line with the priming fluid;
determining a distinct pressure profile indicative of a measurement of current pressure generated during the priming of the subject administration line with the at least one fluid over the course of the priming operation performed therewith;
comparing the distinct pressure profile to the predetermined pressure profile; and
determining, based on a result of the comparison, whether the subject administration line, prior to the priming thereof, contained therein at least one of a liquid as the extant fluid and a gas as the extant fluid.
